(19) United States
(12) Patent Application Publication
(10) Pub. No.: US 2022/0152179 A1
(43) Pub. Date: May 19, 2022

(54) COMBINATION THERAPY WITH OMOMYC AND AN ANTIBODY BINDING PD-1 OR CTLA-4 FOR THE TREATMENT OF CANCER

(71) Applicants: FUNDACIÓ PRIVADA INSTITUT D'INVESTIGACIÓ ONCOLÒGICA DE VALL HEBRON, Barcelona (ES); INSTITUCIÓ CATALANA DE RECERCA I ESTUDIS AVANÇATS, Barcelona (ES); PEPTOMYC, S.L., Barcelona (ES)

(72) Inventors: Laura SOUCEK, Barcelona (ES); Marie-Eve BEAULIEU, Barcelona (ES); Silvia CASACUBERTA-SERRA, Barcelona (ES)

(21) Appl. No.: 17/593,408

(22) PCT Filed: Mar. 18, 2020
(86) PCT No.: PCT/EP2020/057492

§ 371 Date: Sep. 17, 2021

(30) Foreign Application Priority Data
Mar. 19, 2019 (EP) 19382194.9

(51) Int. Cl. A61K 39/00; A61K 39/395; A61P 35/00

(52) U.S. Cl. CPC A61K 39/001152; A61P 35/00; A61K 39/3955

(57) ABSTRACT

The invention relates to a combination of an immuno-oncology agent with Omomyc, a functionally equivalent variant thereof, a conjugate comprising Omomyc or said functionally equivalent variant, a polynucleotide encoding said polypeptides, a vector comprising said polynucleotide and a cell capable of secreting the polypeptide or the conjugate. The invention also relates to pharmaceutical compositions containing the combination of the invention and to their medical uses, particularly their uses in the treatment of cancer.

### FIELD OF THE INVENTION

The present invention relates to the field of cancer and, more particularly, to a combination comprising polypeptides and immuno-oncology agents and its use in medicine, more particularly in the prevention and/or treatment of cancer.

### BACKGROUND OF THE INVENTION

The ideal cancer drug should target a non-redundant function continuously necessary for tumor maintenance, but dispensable for maintenance and function of any normal tissues. Hence, the most common logic is to target gene products that are specifically mutated in cancer, on the basis that these mutant molecules would be the likely “drivers” of the cancer and, perhaps, less critical for normal tissues. For these reasons, much attention has focused on cataloguing recurring lesions in specific cancer types. Unfortunately, there are several problems to this approach. First, most solid human cancers pass through episodes of genomic instability and exhibit a mutational noise that can obscure the “driver” mutations and their attendant effector pathways. Second, cancers are the end result of a process that involves transitions through multiple evolutionary bottlenecks. Each bottleneck may require a specific type of mutation whose function is thereafter dispensable for tumor maintenance and, consequently, not a good therapeutic target after that point in the tumor's evolution.

Myc is a basic helix-loop-helix leucine zipper (b-HLH-LZ) protein involved in growth control and cancer, which operates in a network with the structurally related proteins Max, Mad and Mnt. Myc/Max dimers activate gene transcription and induce cell proliferation or apoptosis. Mad/Max and Mnt/Max complexes act as repressors and cause cell growth arrest and differentiation. All dimers recognize the same DNA consensus site, the CACGTG E-box.

Myc is tightly regulated in normal cells, where its levels are higher in proliferating and lower in non-proliferating cells. Aberrantly high and/or deregulated Myc activity is causally implicated in most cancers and often associated with aggressive, poorly differentiated and angiogenic tumors. The deregulation of Myc expression is due to overexpression through gene amplifications, loss of transcriptional control, impaired degradation or increased stabilization. This results in aberrant proliferation, increased survival, changes in metabolism, angiogenesis and inflammation, all of which represent major hallmarks of cancer. Multiple studies substantiated the crucial role of Myc in governing intracellular and extracellular aspects of tumorigenesis suggesting that targeting its function would be therapeutically valuable.

It is known that down-regulation of Myc by a BET bromodomain inhibitor results in the regression of multiple tumor types. While this approach displays good potential, it presents some limitations such as toxicity and numerous off target effects. Many small molecules disrupting the Myc/Max interaction have displayed low specificity in cells.

A Myc inhibitor, however, has yet to become clinically available and its design presents various caveats: first, Myc is a nuclear transcription factor, which is consequently more difficult to reach than membrane or cytoplasmic molecules; second, Myc does not have an enzymatic “active site” that could be targeted; third, the Myc family comprises 3 different proteins, c-, N and L-Myc, which in certain conditions are functionally redundant, so all of them require simultaneous inhibition. Furthermore, there have been concerns that Myc inhibition would induce serious side effects by inhibiting proliferation of normal tissues. For all these reasons, making a Myc inhibitor drug is challenging.

Omomyc is a dominant-negative MYC mutant comprising the b-HLH-LZ domain of Myc and harboring four amino acid substitutions in the leucine zipper of Myc (Soucek, L. et al., 1998, Oncogene 17, 2463-2472; Soucek, L. et al. (2002), Cancer Res 62: 3507-3510). The amino acid substitutions E61T, E68I, R74Q, and R75N confer altered dimerization specificity to the protein, which retains the ability to bind its natural partner Max and to form homodimers with itself as well as heterodimers with wild type c-, N- and L-Myc.

Because of these properties, Omomyc is able to prevent Myc-dependent gene transactivation functions both in vitro and in vivo by negating the ability of Myc to bind its DNA recognition binding site, the E box. At the same time, Omomyc strongly potentiates Myc-induced apoptosis in a manner dependent on Myc expression level and thereby strengthens Myc transrepression activity. Omomyc thus prevents Myc binding to promoter E-boxes and transactivation of target genes while retaining Miz-1-dependent binding to promoters and transrepression. In the presence of Omomyc, the Myc interactome is channeled to repression and its activity switches from a pro-oncogenic to a tumor-suppressive one.

In EP2801370 A1 it was demonstrated that Omomyc peptide itself is capable of efficiently transducing across the cellular membrane and translocate to the nucleus, wherein it exerts its tumor-suppressive effect.

However, there is still a need in the state of the art to develop novel an improved therapeutic approaches for the treatment of cancer.

### BRIEF SUMMARY OF THE INVENTION

In a first aspect, the invention relates to a combination comprising:
i) a first component selected from the group consisting of:
   a) a polypeptide comprising the sequence SEQ ID NO: 1 or a functionally equivalent variant thereof,
   b) a conjugate comprising a polypeptide comprising the sequence SEQ ID NO: 1 or a functionally equivalent variant thereof and a chemical moiety that facilitates cellular uptake of the polypeptide or of the functionally equivalent variant thereof,
   c) a polynucleotide encoding the polypeptide of a) or the conjugate of b),
   d) a vector comprising the polynucleotide according to c), and
   e) a cell capable of secreting into the medium the polypeptide according to (a) or the conjugate according to b).
and
ii) a second component that is an immuno-oncology agent.

In a second aspect, the invention relates to a pharmaceutical composition comprising a pharmaceutically effective amount of a combination according to the invention and a pharmaceutically acceptable excipient.

In a third aspect, the invention relates to a combination according to the invention or a pharmaceutical composition according to the invention for use in medicine.

In a fourth aspect, the invention relates to a combination according to the invention or a pharmaceutical composition according to the invention for use in the prevention and/or treatment of cancer.

### DESCRIPTION OF THE FIGURES

FIG. 1. Omomyc intranasal administration recruits T cells to the tumor site. Mice bearing KRas^{G12D}-driven NSCLC were treated intranasally with Omomyc 4 times a week for 4 weeks. (A) Omomyc administration induced T cell recruitment to the tumor site as early as 1 week after treatment onset and the T cells remained there throughout the treatment. *p<0.05; **p<0.01. (B) FACS analysis showing that Omomyc induces the recruitment of CD4 T cells to the tumor, particularly activated CD4 T cells that display higher levels of the PD-1 and both PD-1 Tim-3 molecules. Omomyc also induces the expansion of T regulatory cells (Tregs).

FIG. 2. Omomyc systemic administration recruits T cells to the tumor site. Kras/p53 mutated NSCLC MuH-163 cell line was inoculated subcutaneously to syngeneic mice. Mice were treated with Omomyc systemically for 3 weeks. Omomyc recruited more CD3⁺ T cells to the tumor site (A) and significantly more CD4 and CD8 T cells expressing both PD-1 and Tim-3 molecules compared to their vehicle counterparts (B). **p<0.01.

FIG. 3. Omomyc in combination with anti-PD-1 recruits CD4⁺PD-1⁺Tim-3⁻ T cells to the tumor. Mice bearing KRas^{G12D}-driven NSCLC were treated intranasally with Omomyc 4 times a week and once a week with anti-PD-1 (250 μg) intraperitoneally for 4 weeks. Omomyc in combination with anti-PD-1 induced the recruitment of CD4⁺ PD-1⁺Tim-3⁻ T cells to the tumor site.

FIG. 4. Omomyc in combination with anti-PD-1 induces the production of IFN-γ. Combination treatment of Omomyc and anti-PD-1 induced the production of IFN-γ by intratumoral CD4 (A) and CD8 (B) T cells.

FIG. 5. Combination of Omomyc with anti-PD-1 antibody synergistically increases the proportion of healthy lung and recruits T cells to the tumor site. Mice bearing KRas^{G12D}-driven NSCLC were treated intranasally with Omomyc 4 times a week for 4 weeks and with anti-PD-1 antibody once a week intraperitoneally. (A) Animals treated with Omomyc in combination with anti-PD-1 presented increased proportions of heathy lung compared to the vehicles and the treatments alone. (B) Representative transverse planar CT images from each experimental group taken at treatment onset and endpoint. Dark areas correspond to healthy lung and grey areas to affected lung. (C) FACS analysis showed that Omomyc and anti-PD-1 administered in combination induced T cell recruitment to the tumor site, in particular of CD4 T cells and of Th1/Th17 cells. *p<0.05; **p<0.01; *** p<0.0001.

FIG. 6. Combination of Omomyc with anti-CTLA-4 antibody synergistically decreases tumor growth and recruits anti-tumor T cells to the tumor site. Mice bearing KRas^{G12D}-driven NSCLC were treated intranasally with Omomyc 4 times a week for 4 weeks and with anti-CTLA-4 antibody once a week intraperitoneally. (A) Animals treated with Omomyc in combination with anti-CTLA-4 presented decreased tumor growth compared to the vehicles and the treatments alone. The table shows the mean of the tumor growth for every treatment group. (B) FACS analysis showed that Omomyc and anti-CTLA-4 administered in combination induced T cell recruitment to the tumor site, in particular of CD4 T cells and of both CD4 and CD8 PD-1⁺ T cells. *p<0.05; **p<0.01; *** p<0.0001.

FIG. 7. Sequential combination of Omomyc with anti-PD-1 antibody synergistically recruits anti-tumor T cells to the tumor site. Mice bearing KRas^{G12D}-driven NSCLC were treated for 10 days intravenously with Omomyc every 4 days and then with anti-PD-1 antibody once a week intraperitoneally. FACS analysis showed that sequential treatment with Omomyc and then with anti-PD-1 induced T cell recruitment to the tumor site, in particular of CD4 T cells expressing both the PD-1 and Tim-3 molecules and of Th1/Th17 T cells expressing PD-1. *p<0.05; **p<0.01.

FIG. 8. Combination of Omomyc with anti-PD-1 antibody synergistically recruits T cells to the tumor site. Mice bearing KRas^{G12D}/p53-driven NSCLC were treated with Omomyc (intravenously) and anti-PD-1 (intraperitoneally) concomitantly once a week. (A) IHC stainings showed that concomitant treatment of Omomyc and anti-PD-1 significantly recruits T cells to the tumor site. (B) FACS analysis showed that treatment with Omomyc with anti-PD-1 induced overall immune cell recruitment to the tumor site. *p<0.05; **p<0.01.

FIG. 9. High expression of CD3, CD4, IL-17 and IFN-γ correlates with higher survival rates. Representative Kaplan-Meier curves of NSCLC patients taking into account the expression of CD3, CD4, IL-17 and IFN-γ. Tables below graphs show the upper quartile survival. Graphs were done with the Kaplan-Meier Plotter http://kmplot.com/analysis/index.php?p=background.

### DETAILED DESCRIPTION OF THE INVENTION

The present invention relates to the provision of new therapeutic combinations for the prevention and treatment of cancer.

Unless otherwise defined, all technical terms used herein have the same meaning as commonly understood by one of ordinary skill in the art to which this invention belongs.

All the embodiments disclosed in relation to an aspect of the invention are applicable to the other aspects.

Combinations and Pharmaceutical Compositions of the Invention

The definitions provided herewith and in every other aspect of the invention are equally applicable to the whole invention.

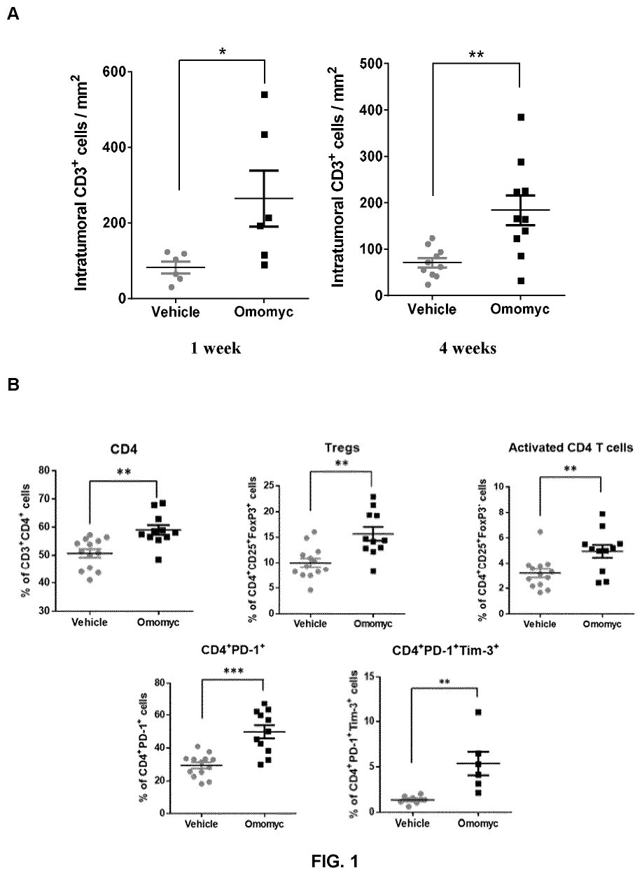

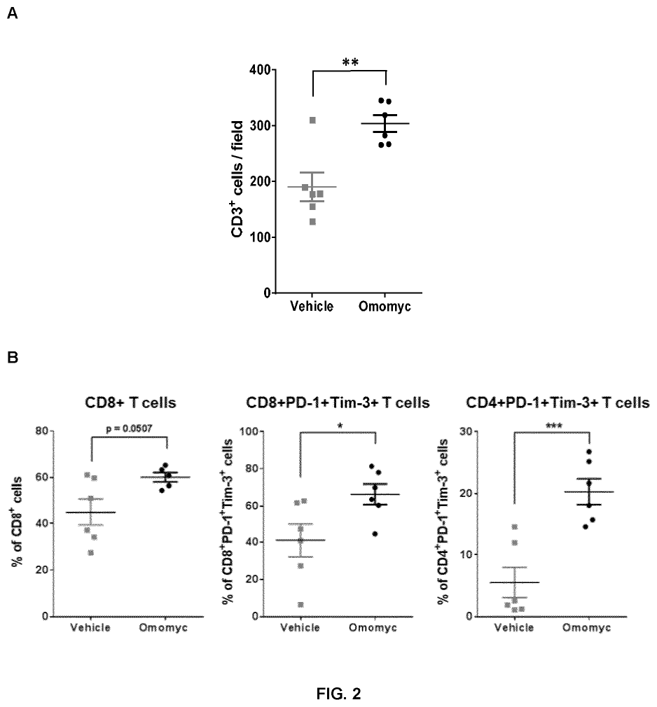

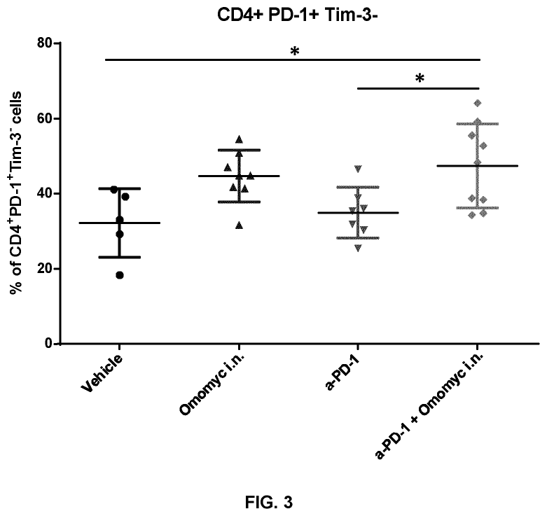

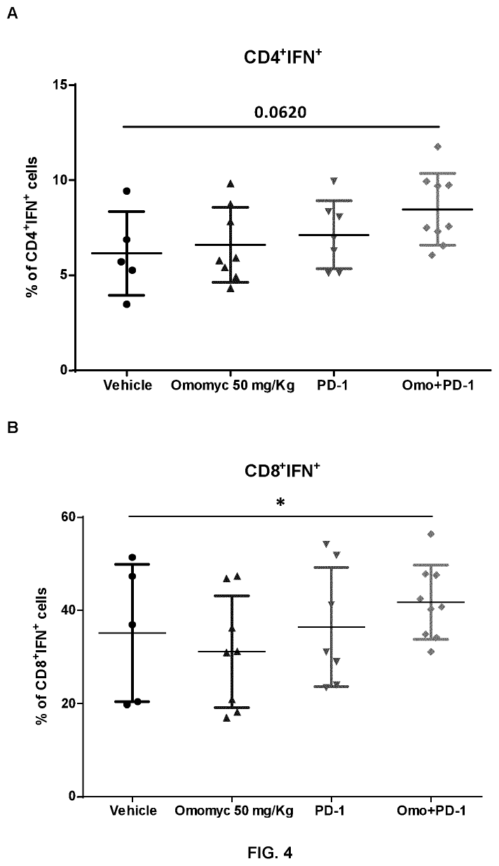

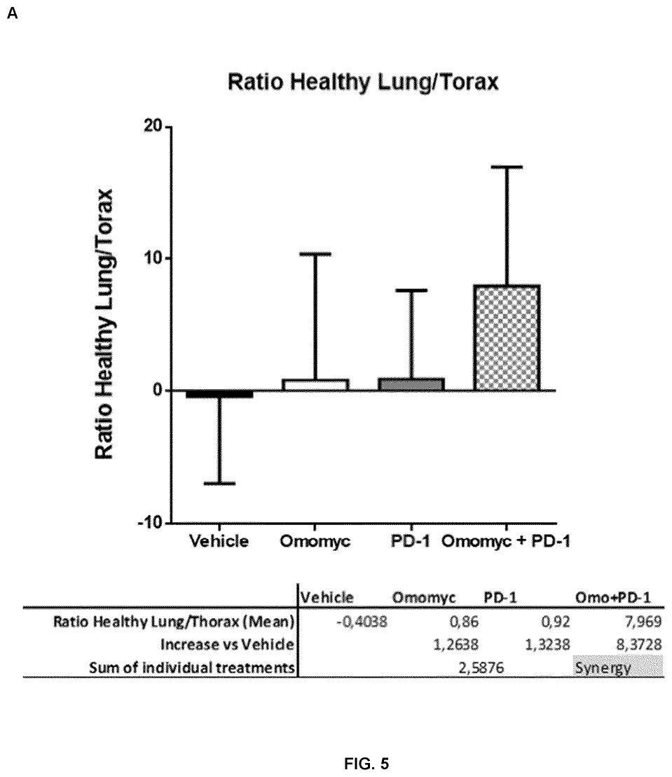

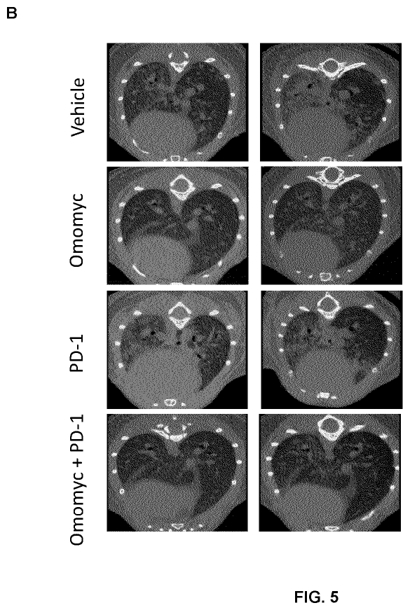

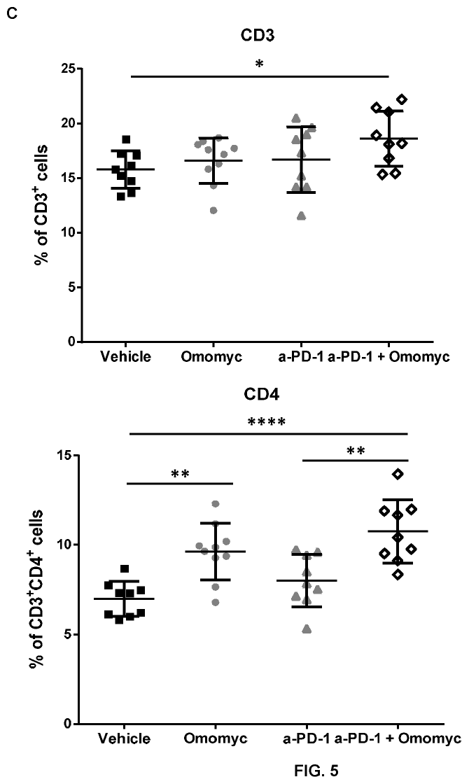

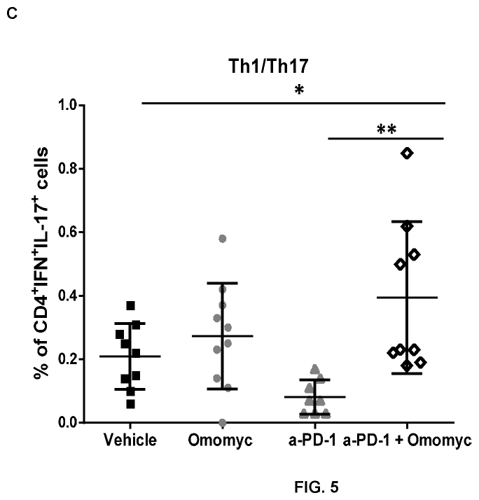

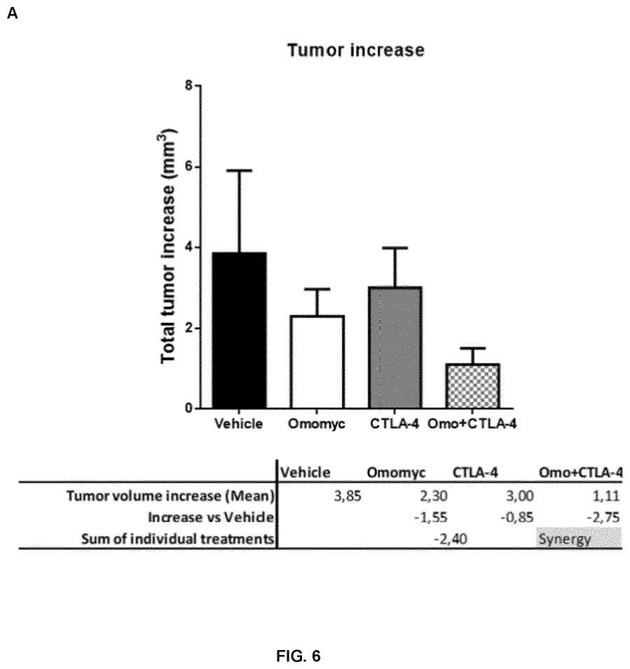

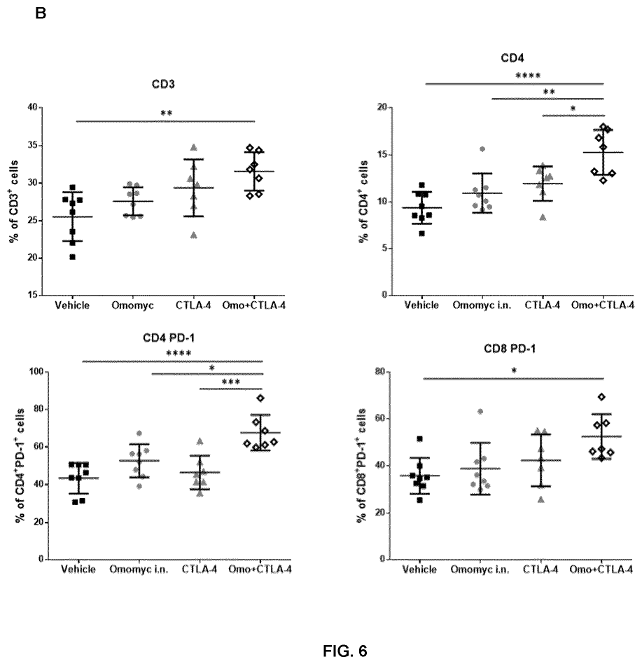

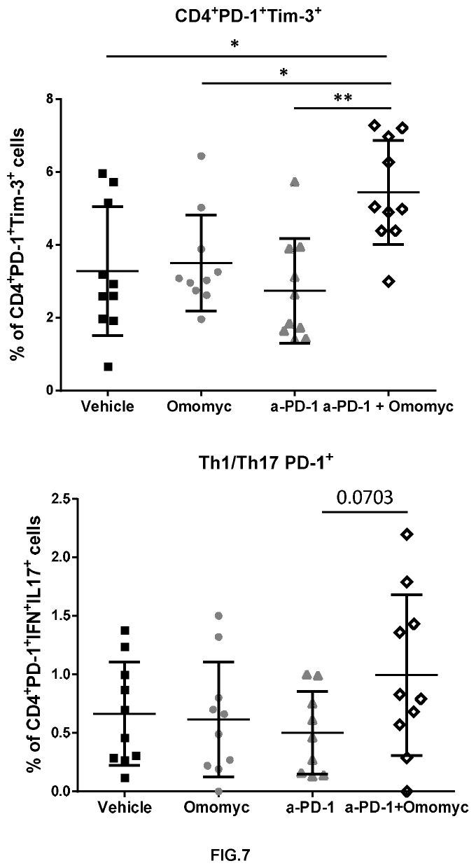

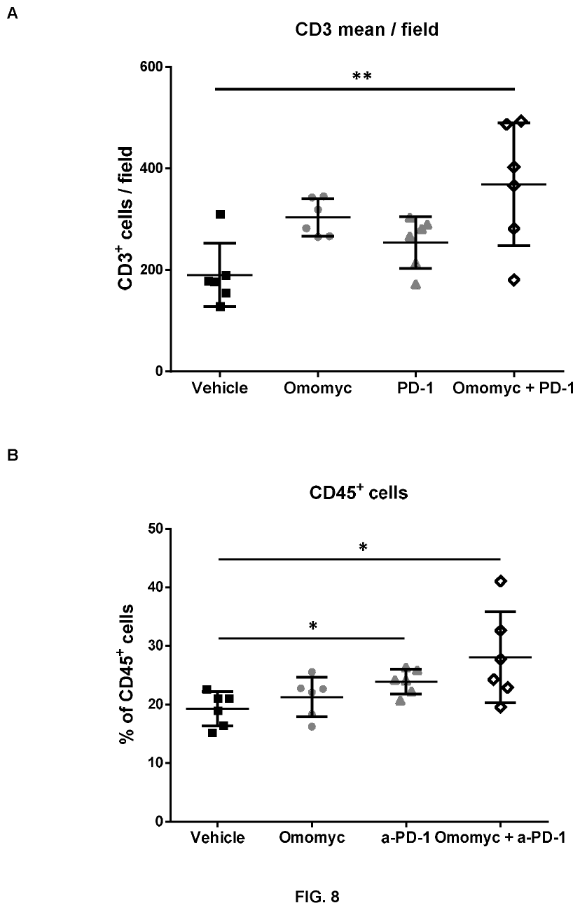

The authors of the present invention have demonstrated that the intranasal and systemic administration of Omomyc recruits T cells to the tumor site (FIGS. 1 and 2). Therefore, Omomyc can be useful in the treatment of cancer in combination with immuno-oncology agents. Furthermore, it has been found that the combination of Omomyc and an immune-oncology agent has a synergistic effect in treating cancers. For example, a combination of Omomyc and an anti-PD-1 therapy significantly increases the recruitment of CD4+ T cells expressing PD-1 but not Tim-3 to the tumor site compared to both the vehicle and anti-PD-1 only treated groups (FIG. 3). Additionally, a combination of Omomyc and anti-PD-1 therapy significantly induces the production of interferon-γ (IFN-γ) by both CD4+ helper and CD8+ cytotoxic intratumoral T cells (FIG. 4) compared to their vehicle counterparts, fact that was not observed either in the Omomyc nor the anti-PD-1 treated groups. The recruitment of T cells to the tumor site translates into a synergistic increase in the proportion of healthy lung when subjects suffering from lung cancer are treated (FIG. 5). This synergistic effect is maintained regardless the route, dose and regime of administration (FIGS. 7 and 8). It has also been found that a combination of Omomyc and an anti-CTLA-4 therapy synergistically decreases tumor growth and recruits anti-tumor T cells to the tumor site (FIG. 6).

Thus, in a first aspect, the invention relates to a combination comprising:
i) a first component selected from the group consisting of:
   a) a polypeptide comprising the sequence SEQ ID NO: 1 or a functionally equivalent variant thereof,
   b) a conjugate comprising a polypeptide comprising the sequence SEQ ID NO: 1 or a functionally equivalent variant thereof and a chemical moiety that facilitates cellular uptake of the polypeptide or of the functionally equivalent variant thereof, and
   c) a polynucleotide encoding the polypeptide of a) or the conjugate of b),
   d) a vector comprising the polynucleotide according to c), and
   e) a cell capable of secreting into the medium the polypeptide according to a) or the conjugate according to b).
and
ii) a second component that is an immuno-oncology agent.

According to the invention, the expression “combination” stands for the various combinations of compounds (i) and (ii), for example in a composition formulated as a single formulation, in a combined mixture composed from separate formulations of each of the components, such as a “tank-mix” which may be combined for joint use as a combined preparation, and in a combined use of the single active ingredients when applied in a sequential manner, i.e., one after the other with a reasonably short period, such as a few hours or days or in simultaneous administration. In the present invention, compound (i) refers to a therapeutically effective amount of a polypeptide comprising the sequence SEQ ID NO: 1 or a functionally equivalent variant thereof, or refers to a conjugate comprising a polypeptide comprising the sequence SEQ ID NO: 1 or a functionally equivalent variant thereof and a chemical moiety that facilitates cellular uptake of the polypeptide or of the functionally equivalent variant thereof, or refers to a polynucleotide encoding the polypeptide or the conjugate, or refers to a vector comprising the polynucleotide, or refers to a cell capable of secreting into the medium the polypeptide or the conjugate. In the present invention, compound (ii) refers to a therapeutically effective amount of an immuno-oncology agent. Preferably, the order of applying the compounds (i) and (ii) is not essential for working the present invention.

The combination may be a kit-of-parts wherein each of the components is individually formulated and packaged.

A combination of compounds (i) and (ii) can be formulated for its simultaneous, separate or sequential administration. Particularly, if the administration is not simultaneous, the compounds are administered in a close time proximity to each other. Furthermore, compounds are administered in the same or different dosage form or by the same or different administration route, e.g. one compound can be administered orally and the other compound can be administered intravenously. Preferably, compound (i) is administered intranasally and compound (ii) is administered systemically, more preferably parenterally, even more preferably intraperitoneally. In another embodiment, compound (i) is administered intravenously and compound (ii) is administered parenterally, even more preferably intraperitoneally.

The combination of the two compounds (i) and (ii) can be administered:
as a combination that is being part of the same medicament formulation, the two compounds being then administered always simultaneously.
as a combination of two units, each with one of the substances giving rise to the possibility of simultaneous, sequential or separate administration.

In a particular embodiment, compound (i) of the combination of the invention is independently administered from compound (ii), i.e. in two units, but at the same time.

In another particular embodiment, compound (i) of the combination of the invention is administered first, and then compound (ii), i.e. the compound (ii) is separately or sequentially administered.

In yet another particular embodiment, compound (ii) of the combination of the invention is administered first, and then compound (i), i.e. the compound (i) is administered separately or sequentially, as defined. If administered separately, compounds (i) and (ii) of the combination of the invention can be administered within a period of time from one another, for example, within 1, 2, 3, 4, 5, 6, 7, 8, 9, 10, 11, 12, 13, 14, 15, 16, 17, 18, 19, 20, 21, 22, 23, or 24 hours from one another. In another embodiment, compounds (i) and (ii) of the combination of the invention can be administered within 1, 2, 3, 4, 5, 6, 7, 8, 9, 10, 11, 12, 13, 14, 15, 16, 17, 18, 19, 20, 21, 22, 23, or 24 days from one another, preferably within 1 days from one another, more preferably within 10 days from one another. In a preferred embodiment, compound (ii) is administered after 10 days of the first administration of compound (i). In an embodiment the administration of the first compound is discontinued before starting the administration of the second compound.

In another aspect, the invention relates to a combination or pharmaceutical composition comprising a synergistically effective amount of a first component according to the first aspect of the invention and an immuno-oncology agent.

In a preferred embodiment, compound (i) of the invention is a polypeptide comprising the sequence SEQ ID NO: 1 or a functionally equivalent variant thereof.

The terms “polypeptide” and “peptide” are used interchangeably herein to refer to polymers of amino acids of any length. The polypeptide of the invention can comprise modified amino acids, and it can be interrupted by non-amino acids. In a preferred embodiment the polypeptide is exclusively formed by amino acids. Preferably the polypeptide that forms item (i) of the combination has a length between 80 and 500 amino acids, more preferable between 80 and 300 amino acids, more preferable between 80 and 250 amino acids, more preferably between 80 and 150, even more preferably between 80 and 130 amino acids, preferably between 90 and 130 amino acids, preferably no more than 125 amino acids, more preferably no more than 100 amino acids. In a preferred embodiment, the polypeptide has a length between 90 and 98 amino acids, preferably between 90 and 95 amino acids, more preferably 91 amino acids.

The term “amino acid” refers to naturally occurring and synthetic amino acids, as well as amino acid analogs and amino acid mimetics that function in a manner similar to the naturally occurring amino acids. Furthermore, the term “amino acid” includes both D- and L-amino acids (stereoisomers). Preferably, the amino acids are L-amino acids.

The term “natural amino acids” or “naturally occurring amino acids” comprises the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine.

As used herein, the term “non-natural amino acid” or “synthetic amino acid” refers to a carboxylic acid, or a derivative thereof, substituted at position “a” with an amine group and being structurally related to a natural amino acid. Illustrative non-limiting examples of modified or uncommon amino acids include 2-aminoadipic acid, 3-aminoadipic acid, beta-alanine, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxy lysine, alio hydroxy lysine, 3-hydroxyproline, 4-hydroxyproline, isodesmosine, alloisoleucine, N-methylglycine, N-methyliso leucine, 6-N-methyl-lysine, N-methylvaline, norvaline, norleucine, ornithine, etc.

The polypeptide of the present invention may also comprise non-amino acid moieties, such as for example, hydrophobic moieties (various linear, branched, cyclic, polycyclic or heterocyclic hydrocarbons and hydrocarbon derivatives) attached to the peptides; various protecting groups which are attached to the compound's terminals to decrease degradation. Suitable protecting functional groups are described in Green and Wuts, “Protecting Groups in Organic Synthesis”, John Wiley and Sons, Chapters 5 and 7, 1991.

Chemical (non-amino acid) groups present in the polypeptide may be included in order to improve various physiological properties such as decreased degradation or clearance; decreased repulsion by various cellular pumps, improve various modes of administration, increased specificity, increased affinity, increased stability, bioavailability, solubility, decreased toxicity and the like.

“Mimetic” include molecules which mimic the chemical structure of a peptidic structure and retain the functional properties of the peptidic structure. Approaches to designing peptide analogs, derivatives and mimetics are known in the art.

In an embodiment the polypeptide of the invention is a polypeptide consisting of sequence SEQ ID NO: 1 or a polypeptide consisting of a functionally equivalent variant of SEQ ID NO: 1, preferably is a polypeptide consisting of the sequence SEQ ID NO: 1. The SEQ ID NO: 1 corresponds to

(SEQ ID NO: 1)
TEENVKRRTHNVLERQRRNELKRSFFALRDQIPELENNEKAPKVVILKK
ATAYILSVQAETQKLISEIDLLRKQNEQLKHKLEQLRNSCA

The polypeptide of sequence SEQ ID NO: 1 corresponds to the Omomyc protein sequence. The term “Omomyc”, as used herein, refers to a polypeptide which consists of a mutated version of the bHLHZip domain of the Myc carrying the E61T, E68I, R74Q and R75N mutations (wherein the numbering of the mutated positions is given with respect to the sequence of Myc region corresponding to amino acids 365-454 of the polypeptide as defined under accession number NP_002458 in the NCBI database, release of Mar. 15, 2015). The sequence of c-Myc provided in the NCBI database under the accession number NP_002458 is shown below (SEQ ID NO: 2), wherein the region from which Omomyc derives is shown underlined:

| (SEQ ID NO: 2) | |
|---|---|
| 1 | MDFFRVVENQ QPPATMPLNV SFTNRNYDLD YDSVQPYFYC DEEENFYQQQ QQSELQPPAP |
| 61 | SEDIWKKFEL LPTPPLSPSR RSGLCSPSYV AVTPFSLRGD NDGGGGSFST ADQLEMVTEL |
| 121 | LGGDMVNQSF ICDPDDETFI KNIIIQDCMW SGFSAAAKLV SEKLASYQAA RKDSGSPNPA |
| 181 | RGHSVCSTSS LYLQDLSAAA SECIDPSVVF PYPLNDSSSP KSCASQDSSA FSPSSDSLLS |
| 241 | STESSPQGSP EPLVLHEETP PTTSSDSEEE QEDEEEIDVV SVEKRQAPGK RSESGSPSAG |
| 301 | GHSKPPHSPL VLKRCHVSTH QHNYAAPPST RKDYPAAKRV KLDSVRVLRQ ISNNRKCTSP |
| 361 | RSSDTEENVK RRTHNVLERQ RRNELKRSFF ALRDQIPELE NNEKAPKVVI LKKATAYILS |
| 421 | VQAEEQKLIS EEDLLRKRRE QLKHKLEQLR NSCA |

Omomyc also contains the M2 domain of c-Myc, having the sequence RQRRNELKRSF (SEQ ID NO: 3) (see Dang and Lee, Mol. Cell. Biol., 1988, 8:4048-4054) (double underlined above), and which corresponds to a nuclear localization signal.

Omomyc is characterized in that it shows increased dimerization capacity with all three oncogenic Myc proteins (c-Myc, N-Myc and L-Myc). Omomyc can derive from the bHLHZip domain of any Myc protein known in the art, provided that the mutations which result in the tumor suppressor effect are preserved. Thus, the Omomyc that can be used in the present invention may derive from any mammal species, including but not being limited to domestic and farm animals (cows, horses, pigs, sheep, goats, dog, cats or rodents), primates and humans. Preferably, the Omomyc protein is derived from human Myc protein (accession number NP_002458, release of Mar. 12, 2019).

The term “Myc”, as used, herein, refers to a family of transcription factors which includes c-Myc, N-Myc and L-Myc. Myc protein activates expression of many genes through binding on consensus sequence CACGTG (Enhancer Box sequences or E-boxes and recruiting histone acetyl-transferases or HATs). However, Myc can also act as a transcriptional repressor. By binding the Miz-1 transcription factor and displacing p300 co-activator, it inhibits expression of Miz-1 target genes. Myc also has a direct role in the control of DNA replication.

The Myc b-HLH-LZ or Myc basic region helix-loop-helix leucine zipper domain refers to a region which determines Myc dimerization with Max protein and binding to Myc-target genes. This region corresponds to amino acids 365-454 of human Myc and is characterized by two alpha helices connected by a loop (Nair, S. K., & Burley, S. K., 2003, Cell, 112: 193-205).

In a preferred embodiment, the polypeptide of the invention is a polypeptide that comprises, consists of or consists essentially of the SEQ ID NO: 4 shown below.

(SEQ ID NO: 4)
MTEENVKRRTHNVLERQRRNELKRSFFALRDQIPELENNEKAPKVVILKK
ATAYILSVQAETQKLISEIDLLRKQNEQLKHKLEQLRNSCA

In this context, “consisting essentially of” means that the specified molecule would not contain any additional sequences that would alter the activity of SEQ ID NO: 4.

Preferably, the polypeptide consists of SEQ ID NO: 4.

The term “functionally equivalent variant”, refers to any polypeptide which results from the insertion or addition of one or more amino acids and/or from the deletion of one or more amino acids and/or from the conservative substitution of one or more amino acids with respect to the polypeptide of SEQ ID NO: 1 and/or which results from the chemical modification of the polypeptide of SEQ ID NO: 1 and which substantially preserves the tumor suppressor activity of the SEQ ID NO: 1. Preferably, the functionally equivalent variant refers to any polypeptide which results from the insertion or addition of one or more amino acids and/or from the deletion of one or more amino acids and/or from the conservative substitution of one or more amino acids with respect to the polypeptide of SEQ ID NO: 1 and which substantially preserves the tumor suppressor activity of SEQ ID NO: 1; more preferably results from the insertion or addition of one or more amino acids with respect to the polypeptide of SEQ ID NO: 1.

The skilled person will understand that the preservation of the tumor suppressor activity requires that the variant can dimerize with Myc and/or its obligate partner p21/p22Max and inhibit Myc activity, that it is capable of translocating across the cell membrane and that it is capable of translocating across the nuclear envelope. In some embodiments, the functionally equivalent variant of the polypeptide of the invention homodimerizes less than Omomyc, or is not forced into homodimers by the formation of disulphide bridge. In particular the disulphide bridge formation in the homodimer form of certain embodiments of the polypeptide of the invention is less than in the polypeptide OmoMyc.

“Less homodimerization”, as used herein, relates to the lower ability of forming obligate homodimers of the polypeptide of the invention even in reducing conditions. In a preferred embodiment, the ability is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% less than the ability of forming homodimers of Omomyc.

Reducing conditions, as used herein, relates to the presence of a reducing agent, a compound that donates an electron to another chemical species in a redox chemical reaction. Illustrative, non-limitative examples of reducing agents are DTT (dithiothreitol), b-mercaptoethanol or TCEP (tris(2-carboxyethyl)phosphine). It is possible that the amount of homodimers is the same in vitro, and that the difference between the functionally equivalent variant and Omomyc is present only in cells in presence of heterodimerization partners where the absence of the disulfide enables a potentially higher formation of heterodimers.

Several assays may be used to determine the homodimerization of a peptide, by way of illustrative non-limitative example by thermal denaturation monitored by Circular dichroism, so dimerization may be detected through folding and thermal stability quantification.

Suitable functionally equivalent variants include polypeptides consisting essentially of the polypeptide of SEQ ID NO: 1. In this context, “consisting essentially of” means that the specified molecule would not contain any additional sequences that would alter the activity of the SEQ ID NO: 1.

In a preferred embodiment, the functionally equivalent variant of SEQ ID NO: 1 is a polypeptide which results from the insertion or addition of one or more amino acids with respect to the polypeptide of SEQ ID NO: 1. In an embodiment, the functionally equivalent variant results from the insertion of less than 10 amino acids, more preferably less than 5 amino acids, more preferably results from the insertion of one amino acid. In a preferred embodiment, results from the insertion of one amino acid that is methionine.

In another embodiment, the functionally equivalent variant of SEQ ID NO: 1 is a polypeptide which results from the deletion of one or more amino acids with respect to the polypeptide of SEQ ID NO: 1. In an embodiment, the functionally equivalent variant results from the deletion of less than 10 amino acids, more preferably less than 5 amino acids, more preferably results from the deletion of one amino acid.

Suitable functional variants of the targeting peptide are those showing a degree of identity with respect to the peptide of SEQ ID NO:1 of about greater than 25% amino acid sequence identity, such as 25%, 30%, 40%, 50%, 60%, 65%, 70%, 75%, 80%, 85%, 90%, 91%, 92%, 93%, 94%, 95%, 96%, 97%, 98% or 99%. The degree of identity between two polypeptides is determined using computer algorithms and methods that are widely known for the persons skilled in the art. The identity between two amino acid sequences is preferably determined by using the BLASTP algorithm as described previously (BLAST Manual, Altschul, S., et al., NCBI NLM NIH Bethesda, Md. 20894, Altschul, S., et al., J. Mol. Biol. 1990; 215: 403-410). In a preferred embodiment, the sequence identity is determined throughout the whole length of the polypeptide of SEQ ID NO: 1 or throughout the whole length of the variant or of both.

The functionally equivalent variants of the polypeptide of the invention may also include post-translational modifications, such as glycosylation, acetylation, isoprenylation, myristoylation, proteolytic processing, etc.

In another embodiment, suitable functional variants of the targeting peptide are those wherein one or more positions within the polypeptide of the invention contain an amino acid which is a conservative substitution of the amino acid present in the protein mentioned above. “Conservative amino acid substitutions” result from replacing one amino acid with another having similar structural and/or chemical properties. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). Selection of such conservative amino acid substitutions is within the skill of one of ordinary skill in the art and is described, for example, by Dordo et al., (J. Mol. Biol, 1999, 217; 721-739) and Taylor et al., (J. Theor. Biol., 1986, 119:205-218).

It will be understood that the functionally equivalent variants of Omomyc contain mutations at positions corresponding to the mutations E61T, E68I, R74Q and R75N found in Omomyc derived from human c-Myc. The position wherein said mutations have to occur in the functionally equivalent variant can be determined by a multiple sequence alignment of different Myc sequences and identified by the alignment of those positions corresponding to positions 61, 68, 74 and 75 within the sequence of Omomyc derived from human c-Myc. In an embodiment, the functionally equivalent variants of Omomyc contain mutations at positions corresponding to the mutations E61T, E68I, R74Q and R75N found in Omomyc derived from human c-Myc.

In another embodiment, the functionally equivalent variants of Omomyc contain mutations at positions corresponding to E61, E68, R74 and R75 within the sequence of Omomyc wherein E61 has been mutated to E61A or E61S; E68 has been mutated to E68L, E68M or E68V; R74 has been mutated to R74N; and R75 has been mutated to R75Q.

A multiple sequence alignment is an extension of pairwise alignment to incorporate more than two sequences at a time. Multiple alignment methods align all of the sequences in a given query set. A preferred multiple sequence alignment program (and its algorithm) is ClustalW, Clusal2W or ClustalW XXL (see Thompson et al. (1994) Nucleic Acids Res 22:4673-4680). Once the sequences of c-Myc from different organisms and of the variant are compared (aligned) as described herein, the skilled artisan can readily identify the positions within each of the sequence corresponding to positions E61T, E68I, R74Q and R75N found in Omomyc and introduce within the Omomyc variant mutations corresponding to the E61T, E68I, R74Q and R75N mutations found in Omomyc derived from human c-Myc.

Suitable assays for determining whether a polypeptide can be considered as a functionally equivalent variant of Omomyc include, without limitation:
Assays which measure the capacity of the polypeptide to form dimeric complexes with Max and Myc, such as the assays based on the expression of a reporter gene as described in Soucek et al. (Oncogene, 1998, 17: 2463-2472) as well as PLA (protein Ligation assay) or Co-immunoprecipitation.
Assays which measure the capacity of the polypeptide to bind to the Myc/Max recognition site within DNA (the CACGTG site), such as the electrophoretic mobility shift assay (EMSA) described in Soucek et al. (supra.)
Assays which measure the capacity to repress Myc-induced transactivation, such as the assay based on the expression of a reporter gene under the control of the DNA binding sites specific for Myc/Max as described by Soucek et al. (supra.).
Assays based on the capacity of the polypeptide to inhibit growth of cells expressing the myc oncogene, as described by Soucek et al. (supra.).
Assays which measure the ability of the polypeptide to enhance myc-induced apoptosis, such as the assays described by Soucek et al. (Oncogene, 1998: 17, 2463-2472). Moreover, any assay commonly known in the art for assessing apoptosis in a cell can be used, such as the Hoechst staining, Propidium Iodide (PI) or Annexin V staining), trypan blue, DNA laddering/fragmentation and TUNEL.

In a preferred embodiment, a polypeptide is considered a functionally equivalent variant of Omomyc if it shows an activity in one or more of the above assays which is at least 10%, 20%, 30%, 40%, 50%, 60%, 70%, 80%, 90% or 100% of the native Omomyc.

In a particular embodiment, the functionally equivalent variant of the polypeptide of SEQ ID NO: 1 comprises the polypeptide of SEQ ID NO: 1, wherein the residue X at position 89 of SEQ ID NO: 1 is not a cysteine. Preferably, the residue X at position 89 of SEQ ID NO: 1 is an aliphatic amino acid, or a sulfured amino acid, or a dicarboxylic amino acid or their amides, or an amino acid having two basic groups, or an aromatic amino acid, or a cyclic amino acid, or a hydroxylated amino acid. More preferably is an amino acid selected from serine, threonine and alanine, preferably selected from serine and alanine. Suitable functionally equivalent variants of SEQ ID NO: 1 having a residue X at position 89 of SEQ ID NO: 1 which is not a cysteine are disclosed in the following table.

| SEQ ID NO | SEQUENCE |
|---|---|
| SEQ ID NO: | TEENVKRRTHNVLERQRRNELKRSFFALRDQIPELENNEKAPKVVILKKATAYILSV |
| 5 | QAETQKLISEIDLLRKQNEQLKHKLEQLRNSXA (wherein X is any aa different |
| | from Cys) |
| SEQ ID NO: | MTEENVKRRTHNVLERQRRNELKRSFFALRDQIPELENNEKAPKVVILKKATAYILS |
| 6 | VQAETQKLISEIDLLRKQNEQLKHKLEQLRNSXA (wherein X is any aa different |
| | from Cys) |
| SEQ ID NO: | TEENVKRRTHNVLERQRRNELKRSFFALRDQIPELENNEKAPKVVILKKATAYILSV |
| 7 | QAETQKLISEIDLLRKQNEQLKHKLEQLRNSSA |
| SEQ ID NO: | MTEENVKRRTHNVLERQRRNELKRSFFALRDQIPELENNEKAPKVVILKKATAYILS |
| 8 | VQAETQKLISEIDLLRKQNEQLKHKLEQLRNSSA |
| SEQ ID NO: | TEENVKRRTHNVLERQRRNELKRSFFALRDQIPELENNEKAPKVVILKKATAYILSV |
| 9 | QAETQKLISEIDLLRKQNEQLKHKLEQLRNSAA |
| SEQ ID NO: | MTEENVKRRTHNVLERQRRNELKRSFFALRDQIPELENNEKAPKVVILKKATAYILS |
| 10 | VQAETQKLISEIDLLRKQNEQLKHKLEQLRNSAA |

Thus, in a preferred embodiment, the functionally equivalent variant of the polypeptide of SEQ ID NO: 1 is selected from the group consisting of SEQ ID NO: 4, SEQ ID NO: 5, SEQ ID NO: 6, SEQ ID NO: 7, SEQ ID NO: 8, SEQ ID NO: 9 and SEQ ID NO: 10.

Additionally, functionally equivalent variants of Omomyc are also capable of transducing cells after the variant is contacted with said cell. It will be understood that functionally equivalent variants of Omomyc contain the protein transducing domain found in native Omomyc or another functional protein transducing domain.

In a preferred embodiment, a polypeptide is considered as a functionally equivalent variant of SEQ ID NO: 1 if it is capable of transducing a target cell at least 10%, 20%, 30%, 40%, 50%, 60%, 70%, 80%, 90% or 100% as efficiently as SEQ ID NO: 1.

Additionally, functionally equivalent variants of SEQ ID NO: 1 are also capable of translocating to the nucleus of the target tumor cell.

In a preferred embodiment, a polypeptide is considered as a functionally equivalent variant of SEQ ID NO: 1 if it is capable of translocating to the nucleus of the target tumor cells at least 10%, 20%, 30%, 40%, 50%, 60%, 70%, 80%, 90% or 100% as efficiently as the SEQ ID NO: 1.

Suitable assays for determining whether a polypeptide is a functionally equivalent variant of SEQ ID NO: 1 in terms of its ability to translocate across the cellular membrane and to the nucleus include double labelling of a cell with a reagent specific for the polypeptide and with a dye which specifically labels the nucleus of the cell (such as DAPI or Hoechst dye). The detection of the polypeptide of the invention can be performed by confocal microscopy or by fluorescence microscopy.

In another preferred embodiment, compound (i) of the invention is a conjugate comprising a polypeptide comprising the sequence SEQ ID NO: 1 or a functionally equivalent variant thereof and a chemical moiety that facilitates cellular uptake of the polypeptide or of the functionally equivalent variant thereof.

The term “conjugate”, as used herein, refers to two or more compounds which are covalently linked together so that the function of each compound is retained in the conjugate.

The term “chemical moiety” refers to any chemical compound containing at least one carbon atom. Examples of chemical moieties include, but are not limited to, any peptide chain enriched in hydrophobic amino acids and hydrophobic chemical moieties.

In preferred embodiments, the conjugate according to the invention comprises at least 1, at least 2, at least 3, at least 4, at least 5, at least 6, at least 7, at least 8, at least 9, at least 10 or more chemical moieties that facilitate cellular uptake of the polypeptide or of the functionally equivalent variant of said polypeptide.

In one embodiment, the chemical moiety that facilitates cellular uptake of the polypeptide is a lipid or a fatty acid.

A fatty acid generally is a molecule comprising a carbon chain with an acidic moiety (e.g., carboxylic acid) at an end of the chain. The carbon chain of a fatty acid may be of any length, however, it is preferred that the length of the carbon chain be of at least 2, 3, 4, 5, 6, 7, 8, 9, 10, 11, 12, 13, 14, 15, 16, 17, 18, 19, 20 or more carbon atoms, and any range derivable therein. In certain embodiments, the length of the carbon chain is from 4 to 18 carbon atoms in the chain portion of the fatty acid. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated. The fatty acid may be branched, though in preferable embodiments of the present invention, it is unbranched. Specific fatty acids include, but are not limited to, linoleic acid, oleic acid, palmitic acid, linolenic acid, stearic acid, lauric acid, myristic acid, arachidic acid, palmitoleic acid, arachidonic acid.

In a preferred embodiment, the chemical moiety that facilitates the cellular uptake of the polypeptide comprising the sequence SEQ ID NO: 1 or a functionally equivalent variant thereof, is a cell penetrating peptide sequence, in which case, the conjugate would comprise a fusion protein comprising the polypeptide comprising SEQ ID NO: 1 or the functionally equivalent variant thereof and the cell penetrating peptide sequence.

The term “fusion protein” relates to proteins generated by gene technology which consist of two or more functional domains derived from different proteins. A fusion protein may be obtained by conventional means, e.g., by means of gene expression of the nucleotide sequence encoding for said fusion protein in a suitable cell. It will be understood that the cell penetrating peptide refers to a cell penetrating peptide which is different from the cell penetrating peptide which forms part of the polypeptide comprising SEQ ID NO: 1 or of the functionally equivalent variant of SEQ ID NO: 1.

The term “cell penetrating peptide sequence” is used in the present specification interchangeably with “CPP”, “protein transducing domain” or “PTD”. It refers to a peptide chain of variable length that directs the transport of a protein inside a cell. The delivering process into cell commonly occurs by endocytosis but the peptide can also be internalized into cell by means of direct membrane translocation. CPPs typically have an amino acid composition that either contains a high relative abundance of positively charged amino acids such as lysine or arginine or has sequences that contain an alternating pattern of polar/charged amino acid and non-polar, hydrophobic amino acids.

Examples of CPPs which can be used in the present invention include, without limitation, the CPP found in Drosophila antennapedia protein (RQIKIWFQNRRMKWKK. SEQ ID NO:13), the CPP found in the herpesvirus simplex 1 (HSV-1) VP22 DNA-binding protein (DAATATRGRSAASRPTERPRAPARSASRPRRPVE, SEQ ID NO:14), the CPP of Bac-7 (RRIRPRPPRLPRPRPRPLPFPRPG; SEQ ID NO: 15), the CPPs of the HIV-1 TAT protein consisting of amino acids 49-57 (RKKRRQRRR, SEQ ID NO: 16), amino acids 48-60 (GRKKRRQRRRTPQ, SEQ ID NO: 17), amino acids 47-57 (YGRKKRRQRRR; SEQ ID NO: 18); the CPP of 5413-PV peptide (ALWKTLLKKVLKAPKKKRKV; SEQ ID NO: 19), the CPP of penetratin (RQIKWFQNRRMKWKK; SEQ ID NO: 20), the CPP of SynB1 (RGGRLSYSRRRFSTSTGR; SEQ ID NO: 21), the CPP of SynB3 (RRLSYSRRRF; SEQ ID NO: 22), the CPP of PTD-4 (PIRRRKKLRRLK; SEQ ID NO: 23), the CPP of PTD-5 (RRQRRTSKLMKR; SEQ ID NO: 24), the CPP of the FHV Coat-(35-49) (RRRRNRTRRNRRRVR; SEQ ID NO: 25), the CPP of BMV Gag-(7-25) (KMTRAQRRAAARRNRWTAR; SEQ ID NO: 26), the CPP of HTLV-II Rex-(4-16) (TRRQRTRRARRNR; SEQ ID NO: 27), the CPP of D-Tat (GRKKRRQRRRPPQ; SEQ ID NO:28), the CPP R9-Tat (GRRRRRRRRRPPQ; SEQ ID NO: 29), the CPP of MAP (KLALKLALKLALALKLA; SEQ ID NO: 30), the CPP of SBP (MGLGLHLLVLAAALQGAWSQPKKKRKV; SEQ ID NO: 31), the CPP of FBP (GALFLGWLGAAGSTMGAWSQPKKKRKV; SEQ ID NO: 32), the CPP of MPG (ac-GALFLGFLGAAGSTMGAWSQPKKKRKV-cya; SEQ ID NO: 33), the CPP of MPG(ENLS) (ac-GALFLGFLGAAGSTMGAWSQPKSKRKV-cya; SEQ ID NO: 34), the CPP of Pep-1 (ac-KETVVWETVVWTEWSQPKKKRKV-cya; SEQ ID NO: 35), the CPP of Pep-2 (ac-KETVVFETVVFTEWSQPKKKRKV-cya; SEQ ID NO: 36), a polyarginine sequence having the structure RN (wherein N is between 4 and 17), the GRKKRRQRRR sequence (SEQ ID NO: 37), the RRRRRRLR sequence (SEQ ID NO: 38), the RRQRRTS KLMKR sequence (SEQ ID NO: 39); Transportan GWTLNSAGYLLGKINLKALAALAKKIL (SEQ ID NO: 40); KALAWEAKLAKALAKALAKHLAKALAKALKCEA (SEQ ID NO: 41); RQIKIWFQNRRMKWKK (SEQ ID NO: 42), the YGRKKRRQRRR sequence (SEQ ID NO: 43); the RKKRRQRR sequence (SEQ ID NO: 44); the YARAAARQARA sequence (SEQ ID NO: 45); the THRLPRRRRRR sequence (SEQ ID NO: 46); the GGRRARRRRRR sequence (SEQ ID NO: 47).

In a preferred embodiment, said cell-penetrating peptide is not the endogenous contained in SEQ ID NO: 1.

In a preferred embodiment, the CPP is the CPP of the HIV-1 TAT protein consisting of amino acids 49-57 (RKKRRQRRR, SEQ ID NO: 16). In another preferred embodiment the CPP is the GRKKRRQRRR sequence (SEQ ID NO: 37) or RRRRRRLR (SEQ ID NO: 38). In another embodiment, the CPP is the GRKKRRQRRR sequence (SEQ ID NO: 37) or RRRRRRRR (SEQ ID NO: 65).

In some embodiments, a CPP is as a CPP as described in WO2019/018898, the content of which is incorporated herein by reference in its entirety.

In one embodiment, the cell-penetrating peptide sequence is fused at the N-terminus of the polypeptide of the invention or of the functionally equivalent variant of said polypeptide. In another embodiment, the cell-penetrating peptide is fused at the C-terminus of the polypeptide of the invention or of the functionally equivalent variant of said polypeptide.

In preferred embodiments, the conjugates or fusion proteins of the combination according to the invention comprise, in addition to the own cell penetrating peptide found in the polypeptide of SEQ ID NO: 1 or of the functionally equivalent variant of said polypeptide, at least 1, at least 2, at least 3, at least 4, at least 5, at least 6, at least 7, at least 8, at least 9, at least 10 or more additional cell penetrating peptides.

Suitable fusion proteins of the invention include the polypeptides Omomyc*TAT and Omomyc*LZArg as defined below:

| Name | SEQ ID NO: | Sequence |
|---|---|---|
| Omomyc*TAT | 11 | MTEENVKRRTHNVLERQRRNELKRSFFALRDQIPELENNEKAPKVVI |
| | | LKKATAYILSVQAETQKLISEIDLLRKQNEQLKHKLEQLRNSCAGRK |
| | | KRRQRRR |
| Omomyc*LZArg | 12 | MTEENVKRRTHNVLERQRRNELKRSFFALRDQIPELENNEKAPKVVI |
| | | LKKATAYILSVQAETQKLISEIDLLRKQNEQLKHKLEQLRNSCARRR |
| | | RRRLR |

Thus, is a preferred embodiment, the fusion protein is the polypeptide selected from SEQ ID NO: 11 and 12.

Suitable assays for determining whether a conjugate preserves the cell membrane translocation capacity of Omomyc include, without limitation, assays which measure the capacity of the conjugate to transduce cells in culture. This assay is based on contacting the conjugate with culture cells and detecting the presence of the conjugate in an intracellular location.

In another preferred embodiment, the conjugate of the combination of the invention additionally comprises a further nuclear localization signal.

The term “nuclear localization signal” (NLS), as used herein, refers to an amino acid sequence of about 4-20 amino acid residues in length, which serves to direct a protein to the nucleus. Typically, the nuclear localization sequence is rich in basic amino acids and exemplary sequences are well known in the art (Gorlich D. (1998) EMBO 5.17:2721-7). In some embodiments, the NLS is selected from the group consisting of the SV40 large T Antigen NLS (PKKKRKV, SEQ ID NO: 48); the Nucleoplasmin NLS (KRPAATKKAGQAKKKK, SEQ ID NO: 49); the CBP80 NLS (RRRHSDENDGGQPHKRRK, SEQ ID NO: 50); the HIV-I Rev protein NLS (RQARRNRRRWE, SEQ ID NO: 51); the HTLV-I Rex (MPKTRRRPRRSQRKRPPT, SEQ ID NO: 52); the hnRNP A NLS (NQSSNFGPMKGGNFGGRSSGPYGGGGQYFKPRNQGGY, SEQ ID NO: 53); the rpL23a NLS (VHSHKKKKIRTSPTFTTPKTLRLRRQPKYPRKSAPRRNKLDHY, SEQ ID NO: 54). In one embodiment of the invention, the nuclear localization signal comprises the motif K (K/R) X (K/R) (SEQ ID NO: 55).

In an even more preferred embodiment, the nuclear localization signal is selected from the group consisting of PKKKRKV (SEQ ID NO: 48), PAAKRVKLD (SEQ ID NO: 56) and KRPAATKKAGQ AKKKK (SEQ ID NO: 49).

In another preferred embodiment, the NLS may be N-terminal or C-terminal to the conjugate or the fusion protein comprising the polypeptide of SEQ ID NO:1 or a functionally equivalent variant thereof.

The skilled person will understand that it may be desirable that the conjugate of the invention further comprises one or more flexible peptides that connect the polypeptide comprising SEQ ID NO: 1 or the functionally equivalent variant thereof, the cell penetrating peptide sequence and/or the NLS. Thus, in a particular embodiment the polypeptide comprising SEQ ID NO: 1 or a functionally equivalent variant thereof is directly connected to the cell penetrating peptide sequence. In another particular embodiment, the polypeptide comprising SEQ ID NO:1 or a functionally equivalent variant thereof is connected to the cell penetrating peptide sequence through a flexible peptide. In an embodiment the polypeptide comprising SEQ ID NO: 1 or the functionally variant thereof is directly connected to the NLS. In another embodiment the polypeptide comprising SEQ ID NO: 1 or a functionally equivalent variant thereof is connected to the NLS through a flexible peptide.

In a particular embodiment the polypeptide of the conjugate according to the invention is directly connected to the cell penetrating peptide sequence and to the NLS.

In one embodiment, the NLS is one of the NLS which appears endogenously in the Myc sequence, such as the M1 peptide (PAAKRVKLD, SEQ ID NO: 56) or the M2 peptide (RQRRNELKRSF, SEQ ID NO: 57).

In another embodiment the additional NLS refers to an NLS which is different to the endogenous NLS found in polypeptide comprising SEQ ID NO: 1 or in the functionally equivalent variant of SEQ ID NO: 1.

In preferred embodiments, the conjugates or fusion proteins according to the invention comprise, in addition to the endogenous NLS found in the polypeptide of the invention or in the functionally equivalent variant thereof, at least 1, at least 2, at least 3, at least 4, at least 5, at least 6, at least 7, at least 8, at least 9, at least 10 NLS.

In another particular embodiment, the polypeptide of the conjugate fore use according to the invention is connected to the cell penetrating peptide sequence through a first flexible peptide linker and to the NLS through a second flexible peptide linker.

As used herein, the term “flexible peptide”, “spacer peptide” or “linker peptide” refers to a peptide that covalently binds two proteins or moieties but which is not part of either polypeptide, allowing movement of one with respect to the other, without causing a substantial detrimental effect on the function of either the protein or the moiety. Thus, the flexible linker does not affect the tumour tracing activity of the polypeptide sequence, the cell penetrating activity of the cell penetrating peptide or the nuclear localization capacity of the NLS.

The flexible peptide comprises at least one amino acid, at least two amino acids, at least three amino acids, at least four amino acids, at least five amino acids, at least six amino acids, at least seven amino acids, at least eight amino acids, at least nine amino acids, at least 10 amino acids, at least 12 amino acids, at least 14 amino acids, at least 16 amino acids, at least 18 amino acids, at least 20 amino acids, at least 25 amino acids, at least 30 amino acids, at least 35 amino acids, at least 40 amino acids, at least 45 amino acids, at least 50 amino acids, at least 60 amino acids, at least 70 amino acids, at least 80 amino acids, at least 90 amino acids, or about 100 amino acids. In some embodiments the flexible peptide will permit the movement of one protein with respect to the other in order to increase solubility of the protein and/or to improve its activity. Suitable linker regions include a poly-glycine region, the GPRRRR sequence (SEQ ID NO: 58) of combinations of glycine, proline and alanine residues.

In a particular embodiment, the conjugates according to the invention comprise a tag bound to the conjugate or to the C-terminal or N-terminal domain of said polypeptide or fusion protein or variant thereof. Said tag is generally a peptide or amino acid sequence which can be used in the isolation or purification of said fusion protein. Thus, said tag is capable of binding to one or more ligands, for example, one or more ligands of an affinity matrix such as a chromatography support or bead with high affinity. An example of said tag is a histidine tag (His-tag or HT), such as a tag comprising 6 residues of histidine (His6 or H6), which can bind to a column of nickel (Ni²⁺) or cobalt (Co²⁺) with high affinity. His-tag has the desirable feature that it can bind its ligands under conditions that are denaturing to most proteins and disruptive to most protein-protein interactions. Thus, it can be used to remove the bait protein tagged with H6 following the disruption of protein-protein interactions with which the bait has participated.

Additional illustrative, non-limitative, examples of tags useful for isolating or purifying the conjugate or the polypeptide comprising SEQ ID NO: 1 or a variant thereof or a fusion protein include Arg-tag, FLAG-tag (DYKDDDDK; SEQ ID NO:59), Strep-tag (WSHPQFEK, SEQ ID NO:60), an epitope capable of being recognized by an antibody, such as c-myc-tag (recognized by an anti-c-myc antibody), HA tag (YPYDVPDYA, SEQ ID NO:61), V5 tag (GKPIPNPLLGLDST, SEQ ID NO:62), SBP-tag, S-tag, calmodulin binding peptide, cellulose binding domain, chitin binding domain, glutathione 5-transferase-tag, maltose binding protein, NusA, TrxA, DsbA, Avi-tag, etc. (Terpe K., Appl. Microbiol. Biotechnol. 2003, 60:523-525), an amino acid sequence such as AHGHRP (SEQ ID NO:63) or PIHDHDHPHLVIHSGMTCXXC (SEQ ID NO:64), β-galactosidase and the like.

The tag can be used, if desired, for the isolation or purification of said fusion protein.

In another preferred embodiment, compound (i) of the invention is a polynucleotide encoding the polypeptide or the fusion protein disclosed above. In a preferred embodiment, compound (i) of the invention is a polynucleotide encoding a polypeptide comprising the sequence SEQ ID NO: 1 or a functionally equivalent variant thereof. In another embodiment, compound (i) of the invention is a polynucleotide encoding a conjugate comprising the polypeptide comprising the sequence SEQ ID NO: 1 or a functionally equivalent variant thereof and a chemical moiety that facilitates cellular uptake of the polypeptide or of the functionally equivalent variant thereof; more preferably is a polynucleotide encoding a fusion protein between the polypeptide comprising the sequence SEQ ID NO: 1 or a functionally equivalent variant thereof and a cell-penetrating peptide sequence.

The terms “polynucleotide”, “nucleic acid” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-stranded, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. In addition to a native nucleic acid molecule, a nucleic acid molecule of the present invention may also comprise modified nucleic acid molecules. As used herein, mRNA refers to an RNA that can be translated in a cell.

In preferred embodiment, the polynucleotide of the invention is an mRNA.

mRNA can be chemically synthesized, can be obtained by means of in vitro transcription or can be synthesized in vivo in the target cell. The nucleotide sequences that form the polynucleotide encoding the conjugate or fusion protein of the invention are in the same correct reading frame for expression thereof.

In a preferred embodiment, component (i) of the combination of the invention is an mRNA encoding for a polypeptide consisting of the sequence SEQ ID NO: 1 or a polypeptide consisting of a functionally equivalent variant of SEQ ID NO: 1 or a polypeptide consisting of SEQ ID NO: 4.

In another embodiment, component (i) of the combination of the invention is a vector comprising a polynucleotide of the invention.

The term “vector”, as used herein, refers to a nucleic acid sequence comprising the necessary sequences so that after transcribing and translating said sequences in a cell a polypeptide encoded by the polynucleotide of the invention is generated. Said sequence is operably linked to additional segments that provide for its autonomous replication in a host cell of interest. Preferably, the vector is an expression vector, which is defined as a vector which, in addition to the regions of the autonomous replication in a host cell, contains regions operably linked to the nucleic acid of the invention and which are capable of enhancing the expression of the products of the nucleic acid according to the invention. The vectors of the invention can be obtained by means of techniques widely known in the art.

Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. Suitable vectors comprising a polynucleotide of the invention are vectors derived from expression vectors in prokaryotes such as pUC18, pUC19, pBluescript and their derivatives, mp18, mp19, pBR322, pMB9, ColE1, pCRI, RP4, phages and “shuttle” vectors such as pSA3 and pAT28, expression vectors in yeasts such as vectors of the 2-micron plasmid type, integration plasmids, YEP vectors, centromeric plasmids and similar, expression vectors in insect cells such as the vectors of the pAC series and of the pVL series, expression vectors in plants such as vectors of the series pIBI, pEarleyGate, pAVA, pCAMBIA, pGSA, pGWB, pMDC, pMY, pORE and similar and expression vectors in superior eukaryote cells based on viral vectors (adenovirus, virus associated to adenovirus as well as retrovirus and, in particular, lentivirus) as well as non-viral vectors such as pSilencer 4.1-CMV (Ambion), pcDNA3, pcDNA3.1/hyg, pHCMV/Zeo, pCR3.1, pEFI/His, pIND/GS, pRc/HCMV2, pSV40/Zeo2, pTRACER-HCMV, pUB6/V5-His, pVAXI, pZeoSV2, pCI, pSVL, pKSV-10, pBPV-1, pML2d and pTDT1. In a preferred embodiment, the polynucleotide of the invention is comprised in a vector selected from the group consisting of pEGFP or pBabe retroviral vectors and pTRIPZ or pSLIK lentiviral vectors.

The vector of the invention may be used to transform, transfect or infect cells that can be transformed, transfected or infected by said vector. Said cells may be prokaryotic or eukaryotic.

The vector preferably comprises the polynucleotide of the invention operationally bound to sequences that regulate the expression of the polynucleotide of the invention. The regulatory sequences of use in the present invention may be nuclear promoters or, alternatively, enhancer sequences and/or other regulatory sequences that increase expression of the heterologous nucleic acid sequence. In principle, any promoter can be used in the present invention provided said promoter is compatible with the cells wherein the polynucleotide is to be expressed. Thus, promoters suitable for realizing the present invention include, but are not necessarily limited to, constitutive promoters such as derivatives of eukaryotic virus genomes such as polyoma virus, adenovirus, SV40, CMV, avian sarcoma virus, hepatitis B virus, the metallothionein gene promoter, the herpes simplex virus thymidine kinase gene promoter, LTR regions of retroviruses, the immunoglobulin gene promoter, the actin gene promoter, the EF-1alpha gene promoter as well as inducible promoters wherein protein expression depends on the addition of a molecule or exogenous signal, such as tetracycline systems, the NFκB/UV light system, the Cre/Lox system and the heat shock genes promoter, the regulable RNA polymerase II promoters described in WO/2006/135436 and tissue-specific promoters.

In another embodiment, component (i) of the combination of the invention is a cell capable of secreting into the medium the polypeptide of the invention or the conjugate of the invention, preferably the polypeptide of the invention or the fusion protein of the invention.

Suitable cells capable of secreting a polypeptide of the invention include without limitation, cardiomyocytes, adipocytes, endothelial cells, epithelial cells, lymphocytes (B and T cells), mastocytes, eosinophils, vascular intima cells, primary cultures of isolated cells of different organs, preferably of cells isolated from Langerhans islets, hepatocytes, leukocytes, including mononuclear leukocytes, mesenchymal, umbilical cord or adult (of skin, lung, kidney and liver), osteoclasts, chondrocytes and other connective tissue cells. Cells of established lines such as Jurkat T cells, NIH-3T3, CHO, Cos, VERO, BHK, HeLa, COS, MDCK, 293, 3T3 cells, C2C12 myoblasts and W138 cells are also suitable. Persons skilled in the art will appreciate that the cells capable of secreting into the medium a polypeptide of the invention may be found forming microparticles or microcapsules so that the cells have a greater useful life in patients. Materials suitable for the formation of microparticles object of the invention include any biocompatible polymeric material which permits continuous secretion of the therapeutic products and which acts as support of the cells. Thus, said biocompatible polymeric material may be, for example, thermoplastic polymers or hydrogen polymers. Among the thermoplastic polymers we have acrylic acid, acrylamide, 2-aminoethyl methacrylate, poly(tetrafluoroethylene-cohexafluorpropylene), methacrylic-(7-cumaroxy) ethyl ester acid, N-isopropyl acrylamide, polyacrylic acid, polyacrylamide, polyamidoamine, poly(amino)-p-xylylene, poly(chloroethylvinylether), polycaprolactone, poly(caprolactone-co-trimethylene carbonate), poly(carbonate urea) urethane, poly(carbonate) urethane, polyethylene, polyethylene and acrylamide copolymer, polyethylene glycol, polyethylene glycol methacrylate, poly(ethylene terephthalate), poly(4-hydroxybutyl acrylate), poly(hydroxyethyl methacrylate), poly(N-2-hydroxypropyl methacrylate), poly(lactic glycolic acid), poly(L-lactic acid), poly(gamma-methyl, Lglutamate), poly(methylmethacrylate), poly(propylene fumarate), poly(propylene oxide), polypyrrole, polystyrene, poly(tetrafluoroethylene), polyurethane, polyvinyl alcohol, polyethylene of ultra-high molecular weight, 6-(p-vinylbenzamide)-hexanoic acid N-pvinybenzyl-D-maltonamide and copolymers containing more than one of said polymers. Among the polymers of hydrogel type we have natural materials of alginate, agarose, collagen, starch, hyaluronic acid, bovine serum albumin, cellulose and their derivatives, pectin, chondroitin sulphate, fibrin and fibroin, as well as synthetic hydrogels such as Sepharose® and Sephadex®.

Compound (ii) of the combination of the invention is an immuno-oncology agent.

As used herein, the term “an immuno-oncology agent” refers to an agent which is effective to enhance, stimulate, and/or up-regulate immune responses in a subject. In some embodiments, the administration of an immuno-oncology agent with a compound (i) of the combination of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies and cytokines. In some embodiments, an antibody is a monoclonal antibody. In some embodiments, a monoclonal antibody is humanized or human.

In some embodiments, an immuno-oncology agent is a cytokine.

“Cytokines” are understood as peptides of different sizes and molecular weights which synthesize the cells of the immune system for the purpose of regulating the immune response, and they can be hormones, growth factors, necrosis factors, chemokines, etc. They can be of natural origin or from recombinant cell cultures and biologically active equivalents of natural sequence cytokines. Exemplary cytokines can be cytokines that inhibit T cell activation such as IL-6, IL-10, TGF-β, VEGF, and other immunosuppressive cytokines; or a cytokine that stimulates T cell activation, for stimulating an immune response. Their conjugation with antibodies gives rise to immunocytokines. In some embodiments, a cytokine is recombinant human interleukin 15 (rhIL-15), recombinant human interleukin 12 (rhIL-12) such as NM-IL-12 (Neumedicines, Inc.) or heterodimeric IL-15 (hen-15, Novartis/Admune), a fusion complex composed of a synthetic form of endogenous IL-15 complexed to the soluble IL-15 binding protein IL-15 receptor alpha chain (IL15:sIL-15RA).

In another embodiment, the cytokine is selected from the group consisting of IL2, IL7, IL12, IL15, IL21, IL1, IL3, IL4, IL5, IL6, IL8, CXCL8, IL9, II, 10, ILI I, IL13, IL 14, IL16, IL 17, IL 18, IL19, IL20, IL22, IL23, IL25, IL26, IL27, IL28, IL29, IL30, 11,31, 1L32, IL33, 11,35, IL36, GM-CSF, IFN-gamma, IL-1 alpha/IL-IFI, IL-1 beta/IL-IF2, IL-12 p70, IL-12/IL-35 p35, IL-13, IL-17/1L-17A, IL-17A/F Heterodimer, IL-17F, IL-18/IL-1F4, 1L-23, IL-24, IL-32, TL-32 beta, IL-32 gamma, iL-33, LAP (TGF-beta 1), Lymphotoxin-alpha/TNF-beta, TGF-beta, TNF-alpha, TRANCE/TNFSFI I/RANK L and any combination thereof.

In a preferred embodiment, the immuno-oncology agent is not a cytokine. Therefore, in a preferred embodiment, cytokines are excluded from the scope of the present invention. Preferably, the cytokines excluded from the present invention are TNF factor alpha, INF-gamma, GM-GSF factor and IL-2.

In another preferred embodiment, cytokines are excluded from the scope of the present invention only when component (i) of the combination is component (i)(a) or (i)(b). Therefore, in a embodiment, if component (i) of the combination is a polypeptide comprising the sequence SEQ ID NO: 1 or a functionally equivalent variant thereof or a conjugate comprising a polypeptide comprising the sequence SEQ ID NO: 1 or a functionally equivalent variant thereof and a chemical moiety that facilitates cellular uptake of the polypeptide or of the functionally equivalent variant thereof, then the immuno-oncology agent is not a cytokine, preferably is not a cytokine selected from the group consisting of are TNF factor alpha, INF-gamma, GM-GSF factor and IL-2.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6.

Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, a combination of a compound (i) of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonist of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

The term “cytotoxic T-lymphocyte-associated protein 4” (abbreviated “CTLA-4” and also known as cluster of differentiation 152 (CD152)), as used herein, refers to a protein receptor that functions as an immune checkpoint. CTLA-4 is a member of the immunoglobulin superfamily that is expressed by activated T cells and transmits an inhibitory signal to T cells. CTLA-4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA-4 transmits an inhibitory signal to T cells, whereas CD28 transmits a stimulatory signal. CTLA-4 is also found in regulatory T cells (Tregs) and contributes to their inhibitory function. T cell activation through the T cell receptor and CD28 leads to increased expression of CTLA-4. The CTLA-4 protein is encoded by the CTLA-4 gene in humans (Ensembl ref: ENSG00000163599). Normally, after T-cell activation, CTLA-4 is upregulated on the plasma membrane where it functions to downregulate T-cell function through a variety of mechanisms, including preventing co-stimulation by outcompeting CD28 for its ligand, B7, and also by inducing T-cell cycle arrest (Postow et al (2015) J. Clinical oncology, Vol. 33, pages 1974-1983; Pardoll, D. et al (2012), Nature Reviews Cancer 12, 252-264).

In some embodiments, an immuno-oncology agent is a CTLA-4 antagonist. The term “CTLA-4 antagonist”, as used herein, refers without limitation to any chemical compound or agent or biological molecule that blocks binding of CTLA-4 with its ligands B7-1 and/or B7-2. In the context of the present invention, it is understood that when a subject (e.g. human individual) is being treated with a CTLA-4 antagonist (e.g. CTLA-4 antibody), the CTLA-4 antagonist blocks the binding of (human) CTLA-4 to (human) B7-1 and/or B7-2.

Non-limiting examples of CTLA-4 antagonist compounds currently considered for clinical use in the treatment of cancer include antagonistic antibodies against CTLA-4.

In some embodiments, a CTLA-4 antagonist is an antagonistic CTLA-4 antibody. In some embodiments, an antagonistic CTLA-4 antibody is YERVOY (ipilimumab) or tremelimumab.

Other non-limiting examples of CTLA-4 antagonists include immunoadhesins (also known as fusion proteins), which are compounds capable of specifically binding to CTLA-4 and block its binding to B7-1 and/or B7-2.

The term “Programmed Death-1 (PD-1)” receptor, as used herein, refers to an immune-inhibitory receptor belonging to the CD28 family. In humans, PD-1 is encoded by the PDCD1 gene. PD-1 is expressed predominantly on previously activated T cells in vivo, and binds to two ligands, PD-L1 and PD-L2. The term “PD-1”, as used herein, includes human PD-1 (hPD-1), variants, isoforms, and species homologs of hPD-1, and analogues having at least one common epitope with hPD-1. The complete hPD-1 sequence can be found under GENBANK Accession No. U64863). PD-1 is expressed on immune cells such as activated T cells (including effector T cells), B cells, myeloid cells, thymocytes, and natural killer (NK) cells (Suya Dai et al., (2014) Cellular Immunology, Vol:290, pages 72-79; Gianchecchi et al., (2013), Autoimmun. Rev. 12 1091-1 100).

In some embodiments, an immuno-oncology agent is a PD-1 antagonist. The term “PD-1 antagonist”, as used herein, refers without limitation to any chemical compound or agent or biological molecule (e.g. antibody) that blocks binding of PD-L1 expressed on a cancer cell to PD-1 expressed on an immune cell (T cell, B cell or NKT cell) and/or blocks binding of PD-L2 expressed on a cancer cell to the immune-cell expressed PD-1. In the context of the present invention, it is understood that when a subject (e.g., human individual) is being treated with a PD-1 antagonist (e.g., a PD-1 antibody), the PD-1 antagonist blocks the binding of (human) PD-L1 to (human) PD-1, or blocks binding of (human) PD-L2 to (human) PD-1, and preferably blocks binding of both (human) PD-L1 and PD-L2 to (human) PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

Non-limiting examples of PD-1 antagonists are antibodies against PD-1 (also referred to as PD-1 antibodies or anti-PD-1 antibodies) such as for instance PD-1 monoclonal antibody (mAb), or antigen binding fragment thereof, which specifically binds to PD-1, and preferably specifically binds to human PD-1. The mAb may be a human antibody, a humanized antibody or a chimeric antibody, and may include a human constant region. Non-limiting examples of PD-1 antagonist compounds include PD-1 antibodies such as nivolumab (Opdivo®, Bristol-Myers Squibb), pembrolizumab (Keytruda®, Merck), BGB-A317, and others such as PDR001 (Novartis). Other non-limiting examples of PD-1 antagonists include pidilizumab (Cure Tech), AMP-224 (GlaxoSmithKline), AMP-514 (GlaxoSmithKline), PDR001 (Novartis), and cemiplimab (Regeneron and Sanofi). Further PD-1 antagonists also include any anti-PD-1 antibody described in U.S. Pat. Nos. 8,008,449, 7,521,051 and 8,354,509.

Other non-limiting examples of PD-1 antagonists include immunoadhesins (also known as fusion proteins), which are compounds capable of specifically binding to PD-1 and block its binding to PD-L1. Examples of immunoadhesion molecules that specifically bind to PD-1 are described in WO2010/027827, US2016/0304969, and WO2011/066342. For instance, a non-limiting example of fusion proteins that may be used as PD-1 antagonist in the present invention is AMP-224 (which is recombinant B7-DC Fc-fusion protein composed of the extracellular domain of the PD-1 ligand programmed cell death ligand 2 (PD-L2, B7-DC) and the Fc region of human immunoglobulin (Ig) G1).

The term “antibody” (e.g. PD-1 antibody and CTLA-4 antibody), as used herein, refers to any form of antibody, and fragment(s) thereof, which exhibits the desired biological or binding activity (e.g., block the binding of PD-1 to its ligands or block binding of CTLA-4 to its ligands, as discussed above). Thus, it is used in the broadest sense and specifically covers, but is not limited to, monoclonal antibodies (including full length monoclonal antibodies) and fragments thereof, polyclonal antibodies and fragments thereof, multispecific antibodies (e.g., bispecific antibodies) and fragments thereof, humanized, fully human antibodies and fragment thereof, chimeric antibodies and fragments thereof, and camelized single domain antibodies, and fragments thereof.

In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40L antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals) and talimogene laherparepvec (Imlygic®, BioVex/Amgen, previously known as T-VEC). In some embodiments, an immuno-oncology agent is selected from an oncolytic viral therapy such as pexastimogene devacirepvec (PexaVec/JX-594, SillaJen/formerly Jennerex Biotherapeutics), pelareorep (Reolysin®, Oncolytics Biotech), enadenotucirev (NG-348, PsiOxus, formerly known as ColoAd1), ONCOS-102 (Targovax/formerly Oncos), vaccinia viruses engineered to express beta-galactosidase (beta-gal)/beta-glucoronidase and/or beta-gal/human sodium iodide symporter (hNIS) such as GL-ONC1 (GLV-1h68/GLV-1h153, Genelux GmbH) and adenovirus engineered to express GM-CSF such as CG0070 (Cold Genesys).

In some embodiments, an immuno-oncology agent is selected from JX-929 (SillaJen/formerly Jennerex Biotherapeutics), TG01 and TG02 (Targovax/formerly Oncos), TILT-123 (TILT Biotherapeutics), and VSV-GP (VireTherapeutics).

In some embodiments, an immuno-oncology agent is a T-cell engineered to express a chimeric antigen receptor, or CAR. The T-cells engineered to express such chimeric antigen receptor are referred to as a CAR-T cells. CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). Agonists or activators of TLR8 which may be used in the present invention include motolimod (VTX-2337, VentiRx Pharmaceuticals).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE®) antibody construct activates T cells. In some embodiments, a bispecific T cell engager (BITE®) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BiTE®-activated T cells. In some embodiments, the bystander cells comprise tumor-associated antigen (TAA) negative cancer cells. In some embodiments, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

In another embodiment, the immuno-oncology agent of the combination of the invention is an antagonist of a protein that inhibits T cell activation or an immune checkpoint inhibitor.

The term “checkpoint inhibitor”, as used herein, relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDLI, PDL2, PDI, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDLI, PDL2, PDI, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-LI monoclonal Antibody (Anti-B7-HI; MEDI4736), MK-3475 (PD-1 blocker), Nivolumab (anti-PDI antibody), CT-011 (anti-PDI antibody), BY55 monoclonal antibody, AMP224 (anti-PDLI antibody), BMS-936559 (anti-PDLI antibody), MPLDL3280A (anti-PDLI antibody), MSB0010718C (anti-PDLI antibody), and ipilimumab (anti-CTLA-4 checkpoint inhibitor). Checkpoint protein ligands include, but are not limited to PD-LI, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509). In a preferred embodiment, the checkpoint inhibitor is a CD47 inhibitor.

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

In a preferred embodiment, the antagonist of a protein that inhibits T cell activation is selected from anti-PD-1 and anti-CTLA-4.

In a preferred embodiment, the immune-oncology agent is a CTLA-4 antagonist, preferably a CTLA-4 antibody, more preferably ipilimumab or tremelimumab.

In a more preferred embodiment, the antagonist of a protein that inhibits T cell activation is an anti-PD-1. In a preferred embodiment, the anti-PD-1 is an antibody or an antigen-binding portion thereof, preferably an antibody selected from the group consisting of OPDIVO (nivolumab), KEYTRUDA (pembrolizumab), MEDI-0680 (AMP-514; WO2012/145493), and pidilizumab (CT-011). In another preferred embodiment, the anti-PD-1 is a recombinant protein composed of the extracellular domain of PD-L2 (B7-DC) fused to the Fc portion of IgG1, called AMP-224.

In an embodiment, the combination of the invention is a conjugate between component (i) and component (ii) of the combination of the invention, particularly is a conjugate between a polypeptide comprising the sequence SEQ ID NO: 1 or a functionally equivalent variant thereof and an immuno-oncology agent.

In some embodiments, a conjugation between components (i) and (ii) are via a noncleavable linker. In some embodiments, a conjugation between components (i) and (ii) are via a cleavable linker. Exemplary noncleavable linkers and cleavable linkers are described in U.S. Pat. Nos. 8,088,387, 8,142,784, WO2013075048, U.S. Pat. Nos. 6,630,579, 8,512,707, 9,120,854, 9,023,351, US20160095938, U.S. Pat. No. 9,446,146, WO2005009369, U.S. Pat. Nos. 5,773,001, 6,214,345, U.S. Ser. No. 10/111,954, U.S. Pat. Nos. 8,153,768, 7,829,531, US20160082119, WO2018218004, U.S. Pat. No. 8,568,728, WO2015057699, US20170182181, U.S. Pat. No. 9,198,979, the content of each of which is incorporated herein by reference in its entirety.

In another aspect, the invention relates to a pharmaceutical composition comprising a pharmaceutically effective amount of a combination of the invention together with a pharmaceutically acceptable excipient.

As it is used in the present invention, the expression “pharmaceutical composition” relates to a formulation that has been adapted for administering a predetermined dose of one or several therapeutic useful agents to a cell, a group of cells, an organ, a tissue or an animal in which cell division is uncontrolled, such cancer.

The pharmaceutical composition of the invention contains a pharmaceutically effective amount of a combination according to the invention and a pharmaceutically active carrier. The pharmaceutical composition of the invention comprises a polypeptide comprising the sequence SEQ ID NO: 1, a functionally equivalent variant thereof, a conjugate according to the invention, a polynucleotide encoding the polypeptide or the conjugate, a vector comprising the polynucleotide or a cell capable of secreting into the medium the polypeptide or the conjugate and an immuno-oncology agent. Suitable functionally equivalent variants of the polypeptide of SEQ ID NO: 1, suitable conjugates, fusion proteins, polynucleotides, vectors or cells for use in the pharmaceutical composition according to the invention are as defined above.

The expression “pharmaceutically effective amount”, as used herein, is understood as an amount capable of providing a therapeutic effect, and which can be determined by the person skilled in the art by commonly used means. The amount of the Omomyc polypeptide, of the functionally equivalent variant thereof, of the conjugate, fusion protein, polynucleotide, vector, cell or of the immuno-oncology agent that may be combined in the pharmaceutical compositions according to the invention will vary depending upon the subject and the particular mode of administration. Those skilled in the art will appreciate that dosages may also be determined with guidance from Goodman and Goldman's The Pharmacological Basis of Therapeutics, Ninth Edition (1996), Appendix II, pp. 1707-1711 and from Goodman and Goldman's The Pharmacological Basis of Therapeutics, Tenth Edition (2001), Appendix II, pp. 475-493.

The appropriate dosage of the active principle or principles within the pharmaceutical composition will depend on the type of cancer to be treated, the severity and course of the disease, whether the composition is administered for preventive or therapeutic purposes, previous therapy, the patient's clinical history and response to the peptide or polypeptide, and the discretion of the attending physician.

The amount of polypeptide comprising the sequence SEQ ID NO:1, the functionally equivalent variant thereof, the fusion protein, the conjugate, polynucleotide, vector or cell is suitably administered to the patient at one time or over a series of treatments. Depending on the type and severity of the disease, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day.

In a preferred embodiment, the amount of the first component is of about 3.75 mg/kg, of subject body weight per day, preferably administered four times per week, preferably intranasally administered. In a preferred embodiment, the amount of the first component is of about 8 to 15 mg/m², preferably of 10 to 12 mg/m², more preferably 11.25 mg/m² per day, preferably administered four times per week, preferably intranasally administered.

In a preferred embodiment, the amount of the first component is of about 50 mg/kg, of subject body weight per day, preferably administered twice per week, preferably intravenously administered. In a preferred embodiment, the amount of the first component is of about 100 to 200 mg/m², preferably of 125 to 175 mg/m², preferably of 140 to 160 mg/m², more preferably 150 mg/m² per day, preferably administered twice per week, preferably intravenously administered.

A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

In an embodiment, the combinations or compositions can be administered once a week, twice a week, three times a week, four times a week, five times a week, six times a week or seven times a week. In an embodiment, the combinations or compositions can be administered once a week. In another embodiment, the combinations or compositions can be administered twice per week. In another embodiment, the combinations or compositions can be administered four times a week. In another preferred embodiment, the first component of the combination or composition is administered four times a week and the second component of the combination or composition is administered once a week. In another embodiment, the first component of the combination or composition is administered twice per week and the second component of the combination or composition is administered once a week. Both compounds can be concomitantly administered or sequentially administered. When the compounds are sequentially administered, the administration of the first compound is discontinued before starting with the second compound.

The duration of the treatment can be at least one week, at least two weeks, at least three weeks, at least four weeks, at least five weeks, at least six weeks, at least seven weeks, at least eight weeks, at least nine weeks, at least ten weeks or more. Preferably, the duration of the treatment is at least four weeks. In another embodiment, the duration of the treatment is at least three weeks.

The amount of the immuno-oncology agent depends on the specific agent used and may be about 0.01 mg/kg to about 50 mg/kg and preferably from about 1 mg/kg to about 25 mg/kg, of subject body weight per day, one or more times a day, to obtain the desired therapeutic effect. In a preferred embodiment, the amount of the immune-oncology agent is of about 2.5 mg/kg, of subject body weight per day or 7.5 mg/m² per day, preferably administered once a week, more preferably administered parenterally, even more preferably intraperitoneally. In a preferred embodiment, the amount of the immune-oncology agent is of about 5 mg/kg, of subject body weight per day or 15 mg/m² per day, preferably administered once a week, more preferably administered parenterally, even more preferably intraperitoneally. In another preferred embodiment, the amount of the immune-oncology agent is of about 10 mg/kg, of subject body weight per day, or 30 mg/m² per day, preferably administered once a week, more preferably administered parenterally, even more preferably intraperitoneally.

The pharmaceutical compositions according to the invention, which contain a first component (i) selected from a polypeptide comprising SEQ ID NO:1, a functionally equivalent variant thereof, a fusion protein, a conjugate, a polynucleotide, a vector or a cell according to the invention and a second component (ii) which is an inmuno-oncology agent, may be presented as a single formulation (for example, as a tablet or a capsule comprising a fixed quantity of each one of the components) or can, on the other hand, be presented as separate formulations to be later combined for joint, sequential, or separate administration. The compositions of the invention also include the formulation as a kit-of-parts wherein the components are formulated separately but are packaged in the same container. Those skilled in the art will appreciate that the formulation of the different components in the pharmaceutical composition according to the invention may be similar, in other words, similarly formulated (in tablets or pills), which allows their administration by the same route. In the case where the different components of the invention are formulated separately, the two components can be presented in a blister. Each blister contains the drugs that must be consumed during the day. If the drugs must be administered several times a day, the drugs corresponding to each administration can be placed in different sections of the blister, preferably recording in each section of the blister the time of day when they should be administered. Alternatively, the components of the composition of the invention can be formulated differently so that the different components are differently administered. Thus, it is possible that the first component is formulated as a tablet or capsule for its oral administration and the second component is formulated for its intravenous administration or vice versa. The ratio between the components that are part of the combinations or pharmaceutical compositions according to the invention can be adjusted by the skilled person depending on the antitumor agent used in each particular case, as well as of the desired indication. Thus, the invention envisages compositions wherein the ratio between the quantities of component (i) and component (ii) can range from 50:1 to 1:50, in particular from 20:1 to 1:20, from 1:10 to 10:1, or from 5:1 to 1:5. In a more particular embodiment, the ratio between quantities ranges from 1:1 to 1:5, preferably from 1:1 to 1:3. In a more preferred embodiment, the ratio ranges from 1:1 to 1:1.5, preferably from 1:1.3 to 1:1.4, more preferably 1:1.34. In another preferred embodiment, the ratio ranges from 1:1 to 1:2.8, preferably from 1:2.6 to 1:2.7, more preferably 1:2.67. In another particular embodiment, the ratio between quantities ranges from 30:1 to 5:1, preferably from 30:1 to 8:1, more preferably from 25:1 to 15:1, more preferably from 20:1 to 10:1. In an embodiment, the ratio is 20:1. In another embodiment, the ratio is 10:1. Preferably these ratios are w/w ratios.

The components of the pharmaceutical composition or the combination of the invention can be administered simultaneously. “Simultaneous administration” encompasses coadministration of the two therapeutic agents, regardless of the relative frequencies or timing of the administration of the respective agents. Thus, simultaneous administration encompasses the coadministration of the two therapeutic agents at the same time and at the same frequencies of administration. In addition, simultaneous administration refers to the coadministration of the two therapeutic agents, in which one agent is administered more frequently than the other(s). In addition, simultaneous administration refers to the coadministration of the two therapeutic agents, in which one agent is administered only once during the administration of the other agent(s).

In an embodiment component (i) is administered intranasally, In another embodiment component (i) is administered intravenously. In another embodiment, component (ii) is administered parenterally, particularly intraperitoneally.

In a preferred embodiment component (i) of the combination or pharmaceutical composition of the invention is administered intranasally, whereas the immuno-oncology agent is administered parenterally, particularly intraperitoneally or intravenously. For intranasal administration the preferred dose of component (i) of the combination or composition of the invention, preferably the preferred dose of the polypeptide or functionally equivalent variant thereof, fusion protein or conjugate ranges from 0.01 to 250 mg/Kg which can be administered in single or multiple doses, more preferable between 0.1 to about 100 mg/kg per day. The preferred dose of the immuno-oncology agent for intraperitoneal administration is 0.01 to 150 mg/Kg, more preferable between 0.1 to 100 mg/Kg.

In another embodiment component (i) of the combination or pharmaceutical composition of the invention is administered intravenously, whereas the immune-oncology agent is administered parenterally, particularly intraperitoneally or intravenously.

The pharmaceutical composition of the invention can also contain one or several additional compounds for the prevention and/or treatment of pathologies in which there is an uncontrolled cell division, such as cancer. Said additional compounds, such as antitumoral agents can form part of the pharmaceutical composition as independent entities. In a preferred embodiment, the combinations or pharmaceutical compositions of the invention comprise one or more antitumoral agents selected from the group consisting of a cytotoxic agent, an antiangiogenic agent, an antimetastatic agent and an antiproliferative agent.

The pharmaceutical composition of the invention also contain one or several additional pharmaceutically acceptable excipients. “Pharmaceutically acceptable excipient” is understood a therapeutically inactive substance said to be used for incorporating the active ingredient and which is acceptable for the patient from a pharmacological/toxicological point of view and for the pharmaceutical chemist who manufactures it from a physical/chemical point of view with respect to the composition, formulation, stability, acceptation of the patient and bioavailability. The excipient can be a carrier. As used herein “carrier” is meant any substance that serves to improve the delivery and the effectiveness of the active principle within the pharmaceutical composition. In a preferred embodiment, the carrier does not allow direct delivery of component (i) and/or (ii) to the cytoplasm of the cells, i.e. the carrier is not capable of fusing with the plasmatic membrane of the target cells. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the combination or composition. Pharmaceutically acceptable carriers may further comprise minor amounts of auxiliary substances such as wetting or emulsifying agents, preservatives or buffers, which enhance the shelf life or effectiveness of components forming part of the combinations or compositions of the invention. Examples of proper carriers are well known in the literature (see for example Remington's Pharmaceutical Sciences, 19th ed., Mack Publishing Company, Easton, Pa., 1995). Examples of carriers without limitation are a series of saccharide such as lactose, dextrose, sucrose, sorbitol, mannitol, xylitol, erythritol, and maltitol; a series of starch such as corn starch, wheat starch, rice starch, and potato starch; a series of cellulose such as cellulose, methyl cellulose, sodium carboxy methyl cellulose, and hydroxyl propylmethyl cellulose; and a series of filler such as gelatin and polyvinyl pyrrolidone. In some cases, a disintegrants such as cross-linked polyvinyl pyrrolidone, agar, alginic acid, or sodium alginate may be added.

The number and the nature of the pharmaceutically acceptable excipients depend on the desired dosage form. The pharmaceutically acceptable excipients are known by the person skilled in the art (Faulí y Trillo C. (1993) “Tratado de Farmacia Galénica”, Luzán 5, S. A. Ediciones, Madrid). Said compositions can be prepared by means of the conventional methods known in the state of the art (“Remington: The Science and Practice of Pharmacy”, 20th edition (2003) Genaro A. R., ed., Lippincott Williams & Wilkins, Philadelphia, US).

For pharmaceutical compositions comprising an agent that is a nucleic acid molecule, the nucleic acid molecule may be present within any of a variety of delivery systems known to those of ordinary skill in the art, including nucleic acid, and bacterial, viral and mammalian expression systems such as, for example, recombinant expression constructs as provided herein. Techniques for incorporating DNA into such expression systems are well known to those of ordinary skill in the art. The DNA may also be “naked,” as described, for example, in Ulmer et al., Science 259:1745-49, 1993 and reviewed by Cohen, Science 259:1691-1692, 1993. The uptake of naked DNA may be increased by coating the DNA onto biodegradable beads, which are efficiently transported into the cells.

Nucleic acid molecules may be delivered into a cell according to any one of several methods described in the art (see, e.g., Akhtar et al., Trends Cell Bio. 2:139 (1992); Delivery Strategies for Antisense Oligonucleotide Therapeutics, ed. Akhtar, 1995, Maurer et al., Mol. Membr. Biol. 16:129-40 (1999); Hofland and Huang, Handb. Exp. Pharmacol. 137:165-92 (1999); Lee et al., ACS Symp. Ser. 752:184-92 (2000); U.S. Pat. No. 6,395,713; International Patent Application Publication No. WO 94/02595); Selbo et al., Int. J. Cancer 87:853-59 (2000); Selbo et al., Tumour Biol. 23:103-12 (2002); U.S. Patent Application Publication Nos. 2001/0007666, and 2003/077829). Such delivery methods known to persons having skill in the art, include, but are not restricted to, encapsulation in liposomes, by iontophoresis, or by incorporation into other vehicles, such as biodegradable polymers; hydrogels; cyclodextrins (see, e.g., Gonzalez et al., Bioconjug. Chem. 10: 1068-74 (1999); Wang et al., International Application Publication Nos. WO 03/47518 and WO 03/46185); poly(lactic-co-glycolic)acid (PLGA) and PLCA microspheres (also useful for delivery of peptides and polypeptides and other substances) (see, e.g., U.S. Pat. No. 6,447,796; U.S. Patent Application Publication No. 2002/130430); biodegradable nanocapsules; and bioadhesive microspheres, or by proteinaceous vectors (International Application Publication No. WO 00/53722). In another embodiment, the nucleic acid molecules can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives (see also, e.g., U.S. Patent Application Publication No. 2003/0077829).

In a particular embodiment, when the compound according to the invention comprises a nucleic acid, the pharmaceutical composition may be formulated as a composition intended for use in gene therapy; by way of illustration, not limitation, that pharmaceutical composition may contain a viral or nonviral vector, which comprises the suitable polynucleotide or gene construction. By way of illustration and not limitation, said vectors, may be viral, for example, based on retrovirus, adenovirus, etc., or nonviral such as ADN-liposome, ADN-polymer, ADN-polymer-liposome complexes, etc. [see “Nonviral Vectors for Gene Therapy”, edited by Huang, Hung and Wagner, Academic Press (1999)]. Said vectors, which contain the corresponding polynucleotide or gene construction, may be administered directly to a subject by conventional methods. Alternatively, said vectors may be used to transform, or transfect or infect cells, for example, mammal cells, including human, ex vivo, which subsequently will be implanted into a human body or an animal to obtain the desired therapeutic effect. For administration to a human body or an animal, said cells will be formulated in a suitable medium that will have no adverse influence on cell viability.

The combination or pharmaceutical composition of the invention can be administered by any type of suitable route, such as by oral route, topical route, by inhalation or parenteral route so that the pharmaceutically acceptable excipients necessary for the formulation of the desired dosage form will be included. Other routes of administration can be rectally, intracisternally or intravaginally. The preferred route of administration of said combination or pharmaceutical compositions is the endovenous route.

“Oral route” is understood as the pharmaceutical composition incorporated into the organism after deglutition. In a particular embodiment, the pharmaceutical composition of the invention can be in a dosage form suitable for its administration by oral route, whether it is solid or liquid. The dosage forms suitable for their administration by oral route can be tablets, capsules, syrups or solutions, and can contain any conventional excipient known in the art, such as binders, for example syrup, acacia, gelatin, sorbitol or polyvinylpyrrolidone; filling agents, for example lactose, sugar, corn starch, calcium phosphate, sorbitol or glycine; lubricants for compression, for example, magnesium stearate; disintegrating agents, for example starch, polyvinylpyrrolidone, sodium glycolate of starch or microcrystalline cellulose; or pharmaceutically acceptable wetting agents such as sodium lauryl sulfate. The solid oral compositions can be prepared by means of conventional processes of mixing, filling or compressing. Repetitive mixing operations can be used to completely distribute the active agent in those compositions that use high amounts of filling agents. Said operations are conventional in the art. The tablets can be prepared, for example, by means of wet or dry granulation, and optionally coating them according to the processes known in the common pharmaceutical practice, particularly with an enteric coating.

On the other hand, “topical route” is understood as an administration by non-systemic route, and includes the application of a pharmaceutical composition of the invention externally on the epidermis, in the oral cavity and the instillation of said composition into ears, eyes and nose, and in which it does not significantly enter the blood stream. Dosage forms for topical or transdermal administration of a compound of this invention include ointments, pastes, creams, lotions, gels, powders, solutions, sprays, inhalants or patches.

Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In an embodiment, the combination or pharmaceutical composition is administered systemically.

“Systemic route” is understood as the administration by oral route, intravenous route, intraperitoneal route and intramuscular route. The amount of components (i) and (ii) required for the therapeutic or prophylactic effect will naturally vary according to the elected compound, the nature and the severity of the illness that is going to be treated, and the patient.

In another embodiment, the combination or pharmaceutical composition is administered intranasally. In a preferred embodiment, the intranasal administration is performed by instillation or nasal inhalation.

“Inhalation” is understood as the administration by intranasal route and by oral inhalation. The dosage forms suitable for said administration, such as a formulation in aerosol or a meter dosed inhaler can be prepared by means of conventional techniques. In an embodiment the route of administration is the intranasal route.

As it is used herein, the term “parenteral”, includes administration by intravenous route, intraperitoneal route, intramuscular route or subcutaneous route. Subcutaneous, intramuscular and intravenous dosage forms of parenteral administration are generally preferred.

In one embodiment, the combinations or pharmaceutical compositions of the invention can be adapted for their parenteral administration, such as sterile solutions, suspensions or lyophilized products in the appropriate dosage unit form. The combinations or pharmaceutical compositions suitable for its injectable use include sterile aqueous solutions (when they are soluble in water), or dispersions and sterile powders for the extemporaneous preparation of sterile injectable solutions or dispersions. For its administration by intravenous route, some suitable carriers include saline solution buffered with phosphate (PBS). In all the cases, the combination or composition must be sterile, and must be fluid to the point which that there exists easy ability for being injected. It must be stable in the preparation and storage conditions, and must be protected from the contamination action of microorganisms such as bacteria and fungi. The carrier can be a solvent or a dispersion medium which contains, for example, water, ethanol, a pharmaceutically acceptable polyol such as glycerol, propylene glycol, liquid polyethylene glycol and suitable mixtures thereof. Suitable fluidity can be maintained, for example, by means of using a coating such as lecithin, by means of maintaining the particle size required in the case of dispersion and by means of using surfactants. The prevention of the action of the microorganisms can be achieved by means of various antibacterial and antifungal agents, for example, parabens, chlorobutanol, phenol, ascorbic acid, thiomersal, and the like. In most cases, it will be preferable to include isotonic agents, for example, sugars; polyalcohols such as mannitol, sorbitol; or sodium chloride in the composition. The prolonged absorption of the injectable compositions may be caused by the inclusion of an agent which delays the absorption, for example, aluminum and gelatin monostearate.

The injectable sterile solutions can be prepared by incorporating the active compound in the required amount in a suitable solvent with one or a combination of the aforementioned ingredients, as needed, followed by sterilization by filtration through sterile membranes. Generally, the dispersions are prepared by incorporating the active compound in a sterile vehicle containing a basic dispersion medium and the rest of the ingredients required from among those previously listed. In the case of sterile powders for the preparation of injectable sterile solutions, the preferred preparation processes are vacuum drying and lyophilization which give rise to a powder with the active ingredient plus any desired additional ingredient from a previously filtered sterile solution thereof.

The combinations or pharmaceutical compositions of the invention can be suitably administered by means of pulse infusion, for example, with decreasing doses of the composition. Preferably, the dose is administered by means of injections, more preferably intravenous or subcutaneous injections, partly depending if the administration is acute or chronic. In a preferred embodiment, a PD-1 antagonist is administered by infusion.

Alternatively, as mentioned above, the different components of the composition are differently administered.

Thus, in an embodiment component (i) of the combination or composition, preferably the polypeptide or functionally equivalent variant or the conjugate of the invention, is administered intranasally while the immuno-oncology agent is administered systemically.

In another preferred embodiment, component (i) of the combination or composition, preferably the polypeptide or functionally equivalent variant thereof, or the conjugate of the composition, is administered intranasally or by inhalation.

Dosage forms of compositions intended for intranasal and intrapulmonary administration are preferably a liquid, a suspension or a solid. A suspension is a liquid preparation containing solid particles dispersed in a liquid vehicle. The dosage forms are preferably metered. For example, metered drops/sprays mean that the dispenser that includes the drops/spray delivers the drops/spray containing a metered dose (a predetermined quantity) of the composition for use according to the invention.

One preferred dosage form in the context of the intranasal administration route includes nasal drops. Drops are deposited mostly in the posterior portion of the nose and thus removed rapidly into the nasal pharynx. A concern with drops is often how to precisely control the drug's dose which is particularly important for the administration of the composition.

Another intranasal dosage form by which the pharmaceutical composition of the invention can be administered is nasal sprays. Nasal sprays typically contain the conjugate dissolved or suspended in a solution or a mixture of excipients (e.g. preservatives, viscosity modifiers, emulsifiers, buffering agents) in a non-pressurized dispenser. Nasal sprays have several advantages including compactness of the delivery device, convenience, simplicity of use, and accuracy of delivering dosages of 25 to 200 pL. They are deposited in the anterior portion of the nose and cleared slowly into nasal pharynx by mucociliary clearance. The nasal spray as used herein can be a liquid or a suspension.

Another intranasal dosage form is a nasal aerosol. Nasal aerosols differ from nasal sprays by the method of the composition dispensing: in aerosols, a compound is dispensed due to an excess of pressure and releases through a valve. In sprays, a compound is dispensed due to forcing away by a micropump bucket, while the pressure in the vial is similar to atmosphere pressure. Aerosols have similar advantages as sprays.

The composition according to the invention may alternatively preferably be administered by nasal emulsions, ointments, gels, pastes or creams. These are highly viscous solutions or suspensions applied to the nasal mucosa.

Due to the limited volume of the composition that can be efficiently delivered to the nasal mucosa, liquid intranasal dosage forms usually have higher concentrations as the corresponding intravenous dosage forms. When substances become poorly soluble or are instable in liquid form, powders can be used to administer the composition of the invention. Further advantages of powders are that they do not require preservatives and have usually a higher stability as compared to liquid formulations. The main limitation on intranasal powder application is related to its irritating effect on the nasal mucosa.

One dosage form in context of intrapulmonary administration is an inhalation aerosol. Inhalation aerosols are usually packaged under pressure and contain the composition according to the invention which is released upon activation of a valve system into the respiratory tract, in particular the lungs. The released aerosol is a colloid of fine solid particles (suspension) or liquid droplets (solution) in air or another gas. Accordingly, the aerosol may be a solution or a suspension aerosol. The liquid droplets or solid particles have preferably a diameter of less than 100 pm, more preferably less than 10 pm, most preferably less than 1 pm.

Another dosage form in context of intrapulmonary administration is inhalation sprays. Inhalation sprays are typically aqueous based and do not contain any propellant. They deliver the conjugate to the lungs by oral inhalation.

Nebulized inhalation solutions and suspensions may also be used to deliver the conjugate by the intrapulmonary route. Nebulized inhalation solutions and suspensions are typically aqueous-based formulations that contain the composition according to the invention. The nebulized inhalation solutions and suspensions deliver the composition to the lungs by oral inhalation for systemic effects and are used with a nebulizer.

Dry powder inhalation is an alternative to aerosol inhalation. The composition is usually included in a capsule for manual loading or within the inhaler. Dry powders are typically delivered by an inhaler to the lungs by oral inhalation. The dry powders as used herein can be formulated neat. Neat formulations contain the drug alone or quasi-alone e.g. as spry dried powder. The dry powders as used herein can be also formulated with a carrier such as lactose.

Intrapulmonary dosage forms are preferably metered, i.e. are delivered to the lungs in a predetermined quantity.

Devices for intranasal delivery in the context of the present invention include spray pump systems, pipettes for delivering drops, metered-dose spray pumps, nasal pressurized metered-dose inhalers, powder spray systems, breath-actuated powder inhalers and nasal powder insufflators. The intranasal delivery device may be filled with a single dose amount or a multi-dose amount of the intranasal formulation.

Using the intrapulmonary route the conjugate may be administered with a metered dose inhaler. A metered-dose inhaler (MDI) provides a fine mist of conjugate, generally with an aerodynamic particle size of less than 5 pm.

Dry powder inhalers can be alternatively used to deliver the composition intrapulmonary. Dry powder inhalers present powders as single-dose or multidose powders.

Another device for intrapulmonary delivery is a nebulizer including ultrasonic and air jet nebulizers. In ultrasonic nebulizers, ultrasound waves are formed in an ultrasonic nebulizer chamber by a ceramic piezoelectric crystal that vibrates when electrically excited. This generates an aerosol cloud at the solution surface. The aerosol produced by an air jet nebulizer is generated when compressed air is forced through an orifice. A liquid may be withdrawn from a perpendicular nozzle (the Bernoulli Effect) to mix with the air jet which is atomized using baffles to facilitate the formation of the aerosol cloud.

In one embodiment, each of the components of the combination or the pharmaceutical composition of the invention is prepared with carriers which will protect the components, particular component (i), from a rapid elimination from the body, such as a controlled release formulation, including implants and microencapsulated administration systems. Biodegradable biocompatible polymers such as ethylene vinylacetate, polyanhydrides, polyglycolic acid, collagen, polyorthoesters and polylactic acid can be used. The processes for preparing said formulations will be clear for persons skilled in the art. The materials can also be commercially obtained in Alza Corporation and Nova Pharmaceuticals, Inc.

The sustained release compositions also include preparations of crystals suspended in suitable formulations which can maintain the crystals in suspension. These preparations, when they are injected by subcutaneous or intraperitoneal route may produce a sustained release effect. Other compositions also include the components (i) and/or (ii) trapped in liposomes. The liposomes containing such components are prepared by means of known methods such as Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949. In a preferred embodiment, components (i) and/or (ii) are contained in liposomes, preferably both components are contained in liposomes, more preferably in the same liposome.

Despite the fact that Omomyc, functionally equivalent variants thereof, conjugates and fusion proteins of the invention are capable of translocating across biological membranes, it is possible to formulate Omomyc, any of its functionally equivalent variants, conjugates, polynucleotides, vectors or cells in nanoparticles. The nanoparticles may contribute to preserve the integrity of the components in the biological fluids until it reaches the target organ. Moreover, in the case of compositions comprising component (ii) or other antitumor agent, encapsulation of the composition may decrease secondary effects caused by the antitumor agent. In addition, nanoparticles can also be modified so as to include moieties which allow the targeting of the nanoparticle to an organ of interest. In this way, component (i) of the combination or the composition of the invention will be delivered in the proximity of the target organ, facilitating access of component (i) to the interior of the cells where its biological activity is required.

Thus, in another embodiment, component (i) of the combination or composition of the invention is provided forming part of a nanoparticle. In another embodiment, both components of the combination or composition of the invention are provided forming part of a nanoparticle, preferably both components are provided inside the same nanoparticle.

As used herein, the term “nanoparticle” refers to any material having dimensions in the 1-1,000 nm range. In some embodiments, nanoparticles have dimensions in the 2-200 nm range, preferably in the 2-150 nm range, and even more preferably in the 2-100 nm range. Nanoparticles that can be used in the present invention include such nanoscale materials as a lipid-based nanoparticle, a superparamagnetic nanoparticle, a nanoshell, a semiconductor nanocrystal, a quantum dot, a polymer-based nanoparticle, a silicon-based nanoparticle, a silica-based nanoparticle, a metal-based nanoparticle, a fullerene and a nanotube. Molecules can be either embedded in the nanoparticle matrix or may be adsorbed onto its surface, preferably molecules are embedded in the nanoparticle.

In a preferred embodiment, the nanoparticle is a liposome.

Targeted delivery can be achieved by the addition of ligands without compromising the ability of nanoparticles to deliver their content. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with a nanoparticle, and can be conjugated to the nanoparticles by a variety of methods as discussed herein.

Examples of proteins or peptides that can be used to target nanoparticles include transferin, lactoferrin, TGF-β, nerve growth factor, albumin, HIV Tat peptide, RGD peptide, and insulin, as well as others.

It will be understood that the formulations of the invention in a nanoparticle are not intended or are not solely intended for facilitating the access of the components (i) and/or (ii) to the interior of the cell but to protect components (i) and/or (ii) from degradation and/or for facilitating targeting of the nanoparticle to the organ of interest.

In one example, the nanoparticle may be made up of a biodegradable polymer such as poly(butylcyanoacrylate) (PBCA). Examples of elemental nanoparticles include carbon nanoparticles and iron oxide nanoparticles, which can then be coated with oleic acid (OA)-Pluronic®. In this approach, a drug (e.g., a hydrophobic or water insoluble drug) is loaded into the nanoparticle. Other nanoparticles are made of silica.

Nanoparticles can be formed from any useful polymer. Examples of polymers include biodegradable polymers, such as poly(butyl cyanoacrylate), poly(lactide), poly(glycolide), poly-s-caprolactone, poly(butylene succinate), poly(ethylene succinate), and poly(p-dioxanone); poly(ethyleneglycol); poly-2-hydroxyethylmethacrylate (poly(HEMA)); copolymers, such as poly(lactide-co-glycolide), poly(lactide)-poly(ethyleneglycol), poly(poly(ethyleneglycol)cyanoacrylate-cohexadecylcyanoacrylate, and poly [HEMA-co-methacrylic acid]; proteins, such as fibrinogen, collagen, gelatin, and elastin; and polysaccharides, such as amylopectin, a amylose, and chitosan.

Other nanoparticles include solid lipid nanoparticles (SLN). Examples of lipid molecules for solid lipid nanoparticles include stearic acid and modified stearic acid, such as stearic acid-PEG 2000; soybean lecithin; and emulsifying wax. Solid lipid nanoparticles can optionally include other components, including surfactants, such as Epicuron® 200, poloxamer 188 (Pluronic® F68), Brij 72, Brij 78, polysorbate 80 (Tween 80); and salts, such as taurocholate sodium. Agents can be introduced into solid lipid nanoparticles by a number of methods discussed for liposomes, where such methods can further include high-pressure homogenization, and dispersion of microemulsions.

Nanoparticles can also include nanometer-sized micelles. Micelles can be formed from any polymers described herein. Exemplary polymers for forming micelles include block copolymers, such as poly(ethylene glycol) and poly(ε-caprolactone). (e.g., a PEO-b-PCL block copolymer including a polymer of ε-caprolactone and α-methoxy-ω-hydroxy-poly(ethylene glycol)).

In certain embodiments, the properties of the nanoparticles are altered by coating with a surfactant. Any biocompatible surfactant may be used, for example, polysorbate surfactants, such as polysorbate 20, 40, 60, and 80 (Tween 80); Epicuron® 200; poloxamer surfactants, such as 188 (Pluronic® F68) poloxamer 908 and 1508; and Brij surfactants, such as Brij 72 and Brij 78.

Nanoparticles can optionally be modified to include hydrophilic polymer groups (e.g., poly(ethyleneglycol) or poly(propyleneglycol)), for example, by covalently attaching hydrophilic polymer groups to the surface or by using polymers that contain such hydrophilic polymer groups (e.g., poly[methoxy poly (ethyleneglycol) cyanoacrylate-co-hexadecyl cyanoacrylate]). Nanoparticles can be optionally cross linked, which can be particularly useful for protein-based nanoparticles.

In another embodiment, the pharmaceutical composition of the invention is a nanoemulsion. “Nanoemulsion” as used herein means a colloidal dispersion of droplets (or particles) which at least some of the droplets have diameters in the nanometer size range. The nanoemulsions are comprised of omega-3, -6 or -9 fatty acid rich oils in an aqueous phase and thermo-dynamically stabilized by amphiphilic surfactants, which make up the interfacial surface membrane, produced using a high shear microfluidization process usually with droplet diameter within the range of about 80-220 nm.

Therapeutic Uses of the Invention

In an aspect, the invention relates to the combination or the pharmaceutical composition of the invention for use in medicine.

In further aspect, the invention relates to the combination or the pharmaceutical composition of the invention for use in the prevention and/or treatment of cancer.

In another aspect, the invention refers to the combination or the pharmaceutical composition of the invention for the preparation of a medicament for the prevention and/or treatment of cancer.

In another aspect, the invention also refers to a method for the prevention and/or treatment of cancer that comprises administering to a subject in need thereof a therapeutically effective amount of the combination or pharmaceutical composition of the invention.

In another aspect, the invention also refers to a method for the prevention and/or treatment of cancer by recruiting T cells to the tumor site that comprises administering to a subject in need thereof a therapeutically effective amount of the combination or pharmaceutical composition of the invention. In an embodiment, the T cells recruited to the tumor site are activated CD4 T cells, more particularly are CD4⁺PD-1⁺ T cells, even more particularly are CD4⁺PD-1⁺Tim-3⁻ T cells. In another embodiment, the T cells recruited to the tumor site are CD4⁺PD-1⁺Tim-3⁺ T cells. In another embodiment, the T cells recruited to the tumor site are CD8 T cells, more particularly are CD8⁺PD-1⁺ T cells. In another embodiment the T cells recruited to the tumor site are CD3⁺ T cells. In another embodiment, the T cells recruited to the tumor site are CD3⁺ CD4⁺ T cells. In another embodiment, the T cells recruited to the tumor site are Th1/Th17 cells, particularly Th1/Th17 PD-1⁺ cells, more particularly CD4⁺ IFN⁺ IL-17⁺ T cells, even more particularly CD4⁺ PD-1⁺ IFN⁺ IL-17⁺ T cells. In another embodiment, the cells recruited to the tumor site are CD45⁺ cells.

In another aspect, the invention also refers to a method for the prevention and/or treatment of cancer by inducing the expansion of T regulatory cells that comprises administering to a subject in need thereof a therapeutically effective amount of the combination or pharmaceutical composition of the invention.

In another aspect, the invention also refers to a method for the prevention and/or treatment of cancer by inducing the production of IFN-gamma by intratumoral CD4+ and CD8+ cells that comprises administering to a subject in need thereof a therapeutically effective amount of the combination or pharmaceutical composition of the invention.

In a preferred embodiment, the preventive or therapeutic method according to the invention involves the direct use of a combination or composition comprising a polypeptide comprising Omomyc, a functionally equivalent variant thereof, a conjugate or a fusion protein. Thus, in a preferred embodiment, the preventive or therapeutic methods according to the invention do not involve the administration of the nucleic acid encoding a polypeptide comprising Omomyc or the functionally equivalent variant thereof or fusion protein, or the administration of a vector encoding this nucleic acid, or a cell comprising said nucleic acid.

“Prevention” is understood as the administration of a combination or composition of the invention in an initial or early stage of the disease, or to also prevent its onset.

The term “treatment” is used to designate the administration of a combination or composition of the invention to control the progression of the disease before or after the clinical signs have appeared. Control of the progression of the disease is understood as the beneficial or desired clinical results which include but are not limited to reduction of the symptoms, reduction of the duration of the disease, stabilization of pathological conditions (specifically avoiding additional impairment), delaying the progression of the disease, improving the pathological condition and remission (both partial and complete). The control of the progression of the disease also involves a prolongation of survival in comparison to the expected survival if the treatment was not applied. In a preferred embodiment, control of the progression of the disease is measured as healthy lung/thorax volume ratio. In another embodiment, control of the progression of the disease is measured as reduction in tumor volume.

The term “cancer” is referred to a disease characterized by uncontrolled cell division (or by an increase of survival or apoptosis resistance), by the ability of said cells to invade other neighbouring tissues (invasion) or by the spread to other areas of the body where the cells are not normally located (metastasis) through the lymphatic and blood vessels. Depending on whether or not tumours can spread by invasion and metastasis, they are classified as being either benign or malignant: benign tumours are tumours that cannot spread by invasion or metastasis, i.e., they only grow locally; whereas malignant tumours are tumours that are capable of spreading by invasion and metastasis. The methods according to the present invention are useful for the treatment of local and malignant tumours.

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), hairy cell leukemia, polycythemia vera, lymphoma (e.g., Hodgkin's disease or non-Hodgkin's disease), AIDS-associated leukemias, Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, mendotheliosarcoma, lymphangiosarcoma, lymphangioendotheliosarcoma, synovioma, mesothelioma, Ewing's tumor, leiomyosarcoma, rhabdomyosarcoma, Kaposi's sarcoma, colon carcinoma, pancreatic cancer, breast cancer, biliary tract cancer, esophageal cancer, ovarian cancer, prostate cancer, oral cancer including squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, teratoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, intraepithelial neoplasms including Bowen's disease and Paget's disease, neuroglioma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892).

Other cancers will be known to one of ordinary skill in the art.

In some embodiments, a cancer is melanoma cancer. In some embodiments, a cancer is breast cancer.

In another embodiment, the cancer is glioblastoma.

“Glioblastoma”, also known as glioblastoma and grade IV astrocytoma, is the most common and most aggressive cancer that begins within the brain.

In a preferred embodiment, the cancer is lung cancer.

The terms “lung cancer” or “lung tumour” refer to the physiological condition in mammals characterized by unregulated cell growth in tissues of the lung. The term lung cancer is meant to refer to any cancer of the lung and includes non-small cell lung carcinomas and small cell lung carcinomas. In an embodiment, the lung cancer is non-small cell lung cancer (NSCLC). In another embodiment, the lung cancer is small cell lung cancer (SCLC).

The term non-small cell lung cancer (NSCLC), as used herein, refers to a group of heterogeneous diseases grouped together because their prognosis and management is roughly identical and includes, according to the histologic classification of the World Health Organization/International Association for the Study of Lung Cancer (Travis W D et al. Histological typing of lung and pleural tumours. 3rd ed. Berlin: Springer-Verlag, 1999):

(i) squamous cell carcinoma (SCC), accounting for 30% to 40% of NSCLC, starts in the larger breathing tubes but grows slower meaning that the size of these tumours varies on diagnosis.

(ii) adenocarcinoma is the most common subtype of NSCLC, accounting for 50% to 60% of NSCLC, which starts near the gas-exchanging surface of the lung and which includes a subtype, the bronchioalveolar carcinoma, which may have different responses to treatment.

(iii) large cell carcinoma is a fast-growing form that grows near the surface of the lung. It is primarily a diagnosis of exclusion, and when more investigation is done, it is usually reclassified to squamous cell carcinoma or adenocarcinoma.

(iv) adenosquamous carcinoma is a type of cancer that contains two types of cells: squamous cells (thin, flat cells that line certain organs) and gland-like cells.

(v) carcinomas with pleomorphic, sarcomatoid or sarcomatous elements. This is a group of rare tumours reflecting a continuum in histologic heterogeneity as well as epithelial and mesenchymal differentiation.

(vi) carcinoid tumour is a slow-growing neuroendocrine lung tumour and begins in cells that are capable of releasing a hormone in response to a stimulus provided by the nervous system.

(vii) carcinomas of salivary gland type begin in salivary gland cells located inside the large airways of the lung.

(viii) unclassified carcinomas include cancers that do not fit into any of the aforementioned lung cancer categories.

In a particular embodiment, the NSCLC is selected from squamous cell carcinoma of the lung, large cell carcinoma of the lung and adenocarcinoma of the lung.

The term small cell lung cancer (SCLC), as used herein, refers to a proliferation of small cells with unique and strict morphological features, containing dense neurosecretory granules which give this tumor an endocrine/paraneoplastic syndrome associated. Most cases arise in the larger airways (primary and secondary bronchi). These cancers grow quickly and spread early in the course of the disease.

In an even more preferred embodiment, the lung cancer is adenocarcinoma, more preferably a KRas-driven lung adenocarcinoma, preferably a cancer associated with a mutation in the KRAS gene. In one embodiment, the mutation in the KRAS gene is a mutation at the glycine at position 12, at the glycine at position 13 or at the glutamine at position 61. In a more preferred embodiment, the mutation is selected from the group consisting of the G12S mutation, the G12V mutation, the G12D mutation, the G13D mutation, the G12C mutation, the G12R mutation, the G12F mutation, the G12I mutation, the G13C mutation, the G13R mutation, or the Q61L mutation. In a preferred embodiment, the mutation is the G12D mutation. In another embodiment, the lung cancer is a KRas^{GD12}/p53-driven lung cancer, preferably a KRas^{GD12}/p53-driven NSCLC.

In an embodiment, the cancer is a primary tumor. The term “primary tumor”, as used herein, refers to a tumor that originated in the location or organ in which it is present and did not metastasize to that location from another location.

In another embodiment, the cancer is a cancer metastasis. In the context of the present invention, “metastasis” is understood as the propagation of a cancer from the organ where it started to a different organ. It generally occurs through the blood or lymphatic system. When the cancer cells spread and form a new tumor, the latter is called a secondary or metastatic tumor. The cancer cells forming the secondary tumor are like those of the original tumor. If a breast cancer, for example, spreads (metastasizes) to the lung, the secondary tumor is formed of malignant breast cancer cells. The disease in the lung is metastatic breast cancer and not lung cancer. The authors of the present invention have also observed that the combination or composition of the invention is capable of decreasing cell proliferation irrespective of whether the cancer shows increased expression or activity of the Myc protein. In a preferred embodiment, the cancer to be prevented or treated is Myc-induced cancer.

In an embodiment, the cancer is a solid tumour.

All combinations of compounds of the invention and types of cancer are included in the present invention.

In some embodiments, the combination or composition of the invention produces an arresting of the growth of the tumor. In some embodiments, the combination or composition of the invention produces a reduction in the tumor size (e.g., volume or mass) by at least 5%, 10%, 25%, 50%, 75%, 90% or 99% relative to the size of the tumor prior to treatment. In some embodiments, the combination or composition of the invention produces the reduction of the quantity of the tumor in the patient by at least 5%, 10%, 25%, 50%, 75%, 90% or 99% relative to the quantity of the tumor prior to treatment.

A “subject”, as used herein, includes any animal that has a cancer or exhibits a symptom of cancer, or is at risk for having a cancer or exhibiting a symptom of cancer. Suitable subjects (patients) include laboratory animals (such as mouse, rat, rabbit, or guinea pig), farm animals, and domestic animals or pets (such as cats or dogs). Non-human primates and, preferably, human patients, are included. Preferably, the subject is a mammal, most preferably a human.

The combinations or compositions for use in the prevention and/or treatment of cancer, may be administered using any amount and any route of administration effective for treating or lessening the severity of a cancer. The exact amount required will vary from subject to subject, depending on the species, age, and general condition of the subject, the severity of the disease or condition, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

In a preferred embodiment, component (i) of the invention, preferably the polypeptide or the functionally equivalent variant thereof, or the conjugate, synergistically interact with the immuno-oncology agent of the combination or composition in treating cancer (to achieve the therapeutic effect).

Particularly, in a more preferred embodiment, the combination or pharmaceutical composition for use in the prevention and/or treatment of cancer is a combination or pharmaceutical composition wherein the polypeptide or the functionally equivalent variant thereof, or the conjugate, is in an amount that synergistically interact with the immuno-oncology agent in treating cancer.

The terms “synergistic effect” or “synergistically interact” are used interchangeably. A synergistic effect is one that is greater than the additive effect that would be predicted by summing the actual effects of the individual agents in vitro. In vivo, a synergistic effect is a physiological effect, and particularly a therapeutic effect, that is greater than the additive effect that would be predicted by summing the actual effects of the individual agents in vivo.

Thus, if two agents are administered, they together provide a measurable physiological effect, and particularly a therapeutic effect, if the actual effect of the agents together is greater than would be predicted by summing the actual therapeutic effects of the individual agents. Particularly, a synergistic effect is provided when a first agent alone provides some measurable effect, a second agent alone provides some measurable effect, and together the two agents provide a measurable effect greater than the effect provided by the sum of both individual agents. More particularly, a synergistic effect is provided when a first agent alone provides no measurable effect, a second agent alone provides some measurable effect, and together the two agents provide a measurable effect greater than the effect provided by the second agent alone. Still more particularly, a synergistic effect is provided when neither a first agent alone nor a second agent alone provide any measurable effect, but together the two agents provide a measurable effect. Since components (i) and (ii) act synergistically, the amount of components (i) and/or (ii) of the combinations or compositions of the invention may be less than that required in a monotherapy utilizing only one of them as a therapeutic agent. Preferably, in these combinations or compositions, a dosage of between 0.01-1.000 μg/kg body weight/day of the one or the other therapeutic agent can be administered.

The amount of the therapeutic agents present in the combinations or compositions may be no more than the amount that would normally be administered in a composition comprising that therapeutic agent as the only active agent. Preferably, the amount of a therapeutic agent in the present compositions will range from about 50% to 100% of the amount normally present in a composition comprising that agent as the only therapeutically active agent. In some embodiments, one therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approvided for dosing per the FDA label insert.

The combination or composition of the invention may also be used in combination with known therapeutic processes, for example, in combination with chemotherapy, radiotherapy, immunotherapy, phototherapy, surgical intervention, hormones, or a combination of these.

In a preferred embodiment, the combination or pharmaceutical composition for use in the treatment and/or prevention of cancer is for the treatment of lung cancer, preferably NSCLC, more preferably KRas-driven cancer, even more preferably KRAS^{G12D}-driven cancer, wherein the first component, preferably a polypeptide comprising the sequence SEQ ID NO: 1, more preferably a polypeptide consisting of SEQ ID NO: 1 is administered intranasally; and wherein the second component, preferably an antagonist of a protein that inhibits T cell activation, more preferably an anti-PD-1 or an anti-CTLA-4, even more preferably an anti-PD-1 antibody or an anti-CTLA-4 antibody, is administered systemically, preferably parenterally, even more preferably intraperitoneally. In a preferred embodiment, the first component is administered four times a week. In a preferred embodiment, the second component is administered once a week. In a more preferred embodiment, the first component is administered four times a week and the second component is administered once a week. In an embodiment the first and second components are administered sequentially, i.e., the administration of the first component is discontinued before starting with the administration of the second component. In a preferred embodiment, the treatment lasts for at least four weeks. In a preferred embodiment, the first and second components are administered in different days. In a preferred embodiment, the first component is administered at day 1, 2, 4 and 5 and the second component is administered at day 3. In a preferred embodiment of the co-administration, the first and second components are administered in different days, preferably the first component is administered at day 1, 2, 4 and 5 and the second component is administered at day 3. In a preferred embodiment, the ratio between the quantities of component (i) and component (ii) can range from 50:1 to 1:50, in particular from 20:1 to 1:20, from 1:10 to 10:1, or from 5:1 to 1:5, preferably from 1:1 to 1:5, more preferably from 1:1 to 1:3. In a more preferred embodiment, the ratio can range from 1:1 to 1:1.5, preferably from 1:1.3 to 1:1.4, more preferably 1:1.34. In another preferred embodiment, the ratio ranges from 1:1 to 1:2.8, preferably from 1:2.6 to 1:2.7, more preferably 1:2.67. These ratios are preferably w/w ratios.

In a preferred embodiment, the combination or pharmaceutical composition for use in the treatment and/or prevention of cancer is for the treatment of lung cancer, preferably NSCLC, more preferably KRas-driven cancer, even more preferably KRAS^{G12D}-driven cancer, preferably KRAS′/p53-driven cancer, wherein the first component, preferably a polypeptide comprising the sequence SEQ ID NO: 1, more preferably a polypeptide consisting of SEQ ID NO: 1 is administered intravenously; and wherein the second component, preferably an antagonist of a protein that inhibits T cell activation, more preferably an anti-PD-1 or an anti-CTLA-4, even more preferably an anti-PD-1 antibody or an anti-CTLA-4 antibody, even more preferably an anti-PD-1 antibody, is administered systemically, preferably parenterally, even more preferably intraperitoneally. In a preferred embodiment, the first component is administered twice a week. In a preferred embodiment, the second component is administered once a week. In a more preferred embodiment, the first component is administered twice a week and the second component is administered once a week. In an embodiment the first and second components are administered sequentially, i.e., the administration of the first component is discontinued before starting with the administration of the second component. In another embodiment, the first and second components are concomitantly administered, preferably once a week. In a preferred embodiment, the treatment lasts for at least three weeks, preferably for at least four weeks. In a preferred embodiment, the first and second components are administered in different days. In a preferred embodiment, the first component is administered at days 2 and 5 and the second component is administered at day 3. In a preferred embodiment of the co-administration, the first and second components are administered in different days, preferably the first component is administered at days 2 and 5 and the second component is administered at day 3. In a more preferred embodiment, the first component is administered during a period of time, preferably at least 5 days, at least 10 days, at least 15 days, more preferably 10 days, before administering the second component. In an embodiment, the administration of the first component is discontinued before starting the administration of the second compound. In a preferred embodiment, the ratio between the quantities of component (i) and component (ii) can range from 50:1 to 1:50, in particular from 20:1 to 1:20, from 1:10 to 10:1, or from 5:1 to 1:5. In another particular embodiment, the ratio between quantities ranges from 30:1 to 5:1, preferably from 30:1 to 8:1, more preferably from 25:1 to 15:1, more preferably from 20:1 to 10:1. In an embodiment, the ratio is 20:1. In another embodiment, the ratio is 10:1. These ratios are preferably w/w ratios.

All the embodiments of the combination of the invention are also applicable to the therapeutic methods of the invention.

Articles of Manufacture and Kits

The disclosure also provides articles of manufacture comprising any one of the combinations or the pharmaceutical compositions disclosed herein, in one or more containers. In some embodiments, the article of manufacture comprises, e.g., a brochure, printed instructions, a label, or package insert directing the user (e.g., a distributor or the final user) to combine and/or use the compositions of the article of manufacture for the prevention and/or treatment of cancer.

In some embodiments, the article of manufacture comprises, e.g., bottle(s), vial(s), cartridge(s), box(es), syringe(s), injector(s), or any combination thereof. In some embodiments, the label refers to use or administration of the combinations or the pharmaceutical compositions in the article of manufacture according to the methods disclosed herein. In some aspects, the label suggests, e.g., a regimen for use, a regimen for treating, preventing, or ameliorating a cancer.

The contents of all cited references (including literature references, patents, patent applications, and websites) that may be cited throughout this application are hereby expressly incorporated by reference in their entirety for any purpose, as are the references cited therein.

All terms as used herein, unless otherwise stated, shall be understood in their ordinary meaning as known in the art. Other more specific definitions for certain terms as used in the present application are as set forth below and are intended to apply uniformly throughout the description and claims unless an otherwise expressly set out definition provides a broader definition. Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. Furthermore, the present invention covers all possible combinations of particular and particular embodiments described herein.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). The term “about” as used in connection with a numerical value throughout the specification and the claims denotes an interval of accuracy, familiar and acceptable to a person skilled in the art. In general, such interval of accuracy is ±15%. Unless defined otherwise, all technical and scientific terms used herein have the same meaning as commonly understood by one of ordinary skill in the art to which this disclosure is related. Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

The invention will be described by way of the following examples which are to be considered as merely illustrative and not limitative of the scope of the invention.

### EXAMPLES

Production and Purification of Omomyc

The Omomyc peptide sequence SEQ ID NO: 4 including a methionine at the N-terminal end was reverse transcribed, codon optimized for expression in E. coli, cloned in a pET3a expression vector (Novagen) and purified from BL21 (DE3) Arabinose-Inducible (Invitrogen®) bacterial strain using protocols adapted from the Max° purification protocol described in J.-F. Naud et al. 2003. J Mol Biol, 326:1577-1595; F.-O. and Mcduff et al. 2009. J Mol Recognit, 22:261-269. The purified construct obtained was the polypeptide of SEQ ID NO: 4. Identity of each purified construct was confirmed by mass spectrometry and by western blot analysis. Omomyc was purified by cationic exchange chromatography and purity was confirmed by mass spectrometry analysis, SDS-PAGE and UV spectroscopy. For the in vivo administrations, an additional purification step was carried out in order to remove endotoxins using the ToxinEraser™ Endotoxin Removal Kit (Genscript). Endotoxin concentrations were quantified using the Pierce® LAL Chromogenic Endotoxin Quantification Kit (Thermo Scientific). Buffer exchange was carried out in Amicon Ultra-15 (MerckMillipore) with a 3 kDa exclusion limit.

Omomyc Intranasal Treatment Increased Recruitment of T Lymphocytes Specifically to the Tumour Site

KRas^{LSL-G12D/+} mice were genotyped by Transnetyx and generation of lung tumors in both males and females was performed as described previously (Jackson, E. L., et al., Genes Dev, 2001. 15(24): p. 3243-8). Animals were maintained in a mixed C57BL/6J×FVBN background. A minimum of 5 mice per time point and condition were randomized and treatment started 14-16 weeks after Adeno-Cre infection once the mice presented detectable tumors by micro-CT. Animals were anesthetized by inhaled isoflurane (AbbVie Farmaceutica S.L.U.) and were intranasally treated with either the Omomyc polypeptide (2.4 mg/kg) or the vehicle (10 mM sodium acetate pH 6.5) in 30 μL total volume four times per week (1101100) during either one or four weeks.

At endpoint, mice were euthanized and lungs were excised and perfused through the trachea with 4% PFA, fixed overnight, transferred to 70% ethanol, embedded in paraffin and 4 μm sections were cut. For CD3 immunofluorescence, antigen retrieval was performed by heating 20 min at 400 W in a microwave in 0.01 M citrate buffer pH 6.0. After blocking 1 hour in 3% BSA plus 0.05% Tween20, slides were incubated overnight at 4° C. with anti-CD3 (Dako A0452) diluted 1/100 in Dako Ready-to-use diluent (Dako S2022). After a PBS wash, slides were incubated with goat anti-rabbit IgG (H+L)-AlexaFluor®488 conjugate (Thermo Fisher Scientific A-11008) and stained with DAPI (Life Technologies D1306) diluted 1/10000, washed with PBS and mounted with Fluorescence Mounting Medium (Dako S3023). Images were taken using a Nikon C2+ confocal microscope and the NIS-elements software. Five pictures of representative tumors per mouse were taken and the mean of CD3+ cells per area is shown.

Immunostaining with anti-CD3 revealed that the Omomyc treatment increased recruitment of T lymphocytes specifically to the tumor site as early as 1 week after treatment onset and the T cells remained there throughout all the treatment (FIG. 1A), evidencing a possible immune contribution as part of the mechanism of action of Omomyc polypeptide.

Omomyc Intranasal Treatment Recruits Activated CD4 T Cells to the Tumor Site

The experimental model and Omomyc treatment are the same as the one described previously.

At endpoint, mice were euthanized and lungs were excised and dissociated using the Mouse Tumor Dissociation Kit (Miltenyi) and stained with conjugated antibodies to analyze the immune cell content by flow cytometry. Prior to staining, death cells were stained with the Fixable Viability Stain 510 (BD Biosciences 564406) following manufacturer's instructions. Then unspecific unions were blocked by incubation with the anti-CD16/32 antibody for 10 minutes at room temperature. For surface staining cells were incubated with the antibodies for 20 minutes at 4° C. in the dark. The antibodies used are listed in Table 1. For the intracellular staining of FoxP3 the FoxP3 Transcription Buffer Set (eBioscience 00-5523-00) was used following manufacturer's instructions. Cells were acquired using a CytoFlex cytometer (Beckman Coulter) and data was analyzed using the CytoExpert 2.0 software (Beckman Coulter).

FIG. 1B shows FACS analysis showing that Omomyc induces the recruitment of CD4 T cells to the tumor and that they are activated. In fact, these cells displayed higher levels of the PD-1 and both PD-1 Tim-3 molecules, suggesting that Omomyc induces an anti-tumor immune response. In addition, Omomyc also induces the expansion of T regulatory cells (Tregs).

Omomyc Systemic Administration Recruits T Cells to the Tumor Site

For the studies with the Kras/p53 syngeneic model, 1×10⁶ MuH-163 cells were inoculated subcutaneously to the dorsal flank of 7-week old female C57BL/6 mice (JANVIER LABS). Once the tumors were established and reached a volume of approximately 100 mm³, mice were randomized into two groups and treated intravenously with the vehicle (PBS pH 7.0) or Omomyc (32 mg/kg) once a week. After three weeks of treatment, mice were euthanized and tumors were resected and cut into two parts. One half of the tumors were then fixed overnight with 4% PFA, transferred to 70% ethanol, embedded in paraffin and 4 μm sections were cut. For CD3 immunofluorescence, antigen retrieval was performed by heating 20 min at 400 W in a microwave in 0.01 M citrate buffer pH 6.0. After blocking 1 hour in 3% BSA plus 0.05% Tween20, slides were incubated overnight at 4° C. with anti-CD3 (Dako A0452) diluted 1/100 in Dako Ready-to-use diluent (Dako S2022). After a PBS wash, slides were incubated with goat anti-rabbit IgG (H+L)-AlexaFluor®488 conjugate (Thermo Fisher Scientific A-11008) and stained with DAPI (Life Technologies D1306) diluted 1/10000, washed with PBS and mounted with Fluorescence Mounting Medium (Dako S3023). Images were taken using a motorized Nikon Tie fluorescence microscope and the NIS-elements software. Four pictures of representative areas of the tumor per mouse were taken and the mean of CD3+ cells per field is shown.

For flow cytometry analysis the other half of the tumors were dissociated using the Mouse Tumor Dissociation Kit (Miltenyi) and stained with conjugated antibodies to analyze the immune cell content by flow cytometry. Prior to staining, death cells were stained with the Fixable Viability Stain 510 (BD Biosciences 564406) following manufacturer's instructions. Then unspecific unions were blocked by incubation with the anti-CD16/32 antibody for 10 minutes at room temperature. For surface staining cells were incubated with the antibodies for 20 minutes at 4° C. in the dark. The antibodies used are listed in Table 1. Cells were acquired using a CytoFlex cytometer (Beckman Coulter) and data was analyzed using the CytoExpert 2.0 software (Beckman Coulter).

Omomyc administration induced T cell recruitment to the tumor site (FIG. 2A). Omomyc recruited more CD8 T cells to the tumor and significantly more CD4 and CD8 T cells expressing both PD-1 and Tim-3 molecules (FIG. 2B).

Omomyc in Combination with Anti-PD-1 Recruits CD4⁺PD-1⁺Tim-3⁻ T Cells to the Tumor

KRas^{LSL-G12D/+} mice were genotyped by Transnetyx and generation of lung tumors in both males and females was performed as described previously (Jackson, E. L., et al., Genes Dev, 2001. 15(24): p. 3243-8). Animals were maintained in a pure C57BL/6 background. A minimum of 5 mice per time point and condition were randomized and treatment started 14-16 weeks after Adeno-Cre infection once the mice presented detectable tumors by micro-CT. Mice were randomized into 4 groups: Vehicle+Isotype Rat IgG2a,k, Omomyc+Isotype Rat IgG2a,k, vehicle+anti-PD-1 and Omomyc+anti-PD-1. For Omomyc treatment, animals were anesthetized by inhaled isoflurane (AbbVie Farmaceutica S.L.U.) and were intranasally treated with either the Omomyc polypeptide (2.4 mg/kg) or the vehicle (PBS pH=7) in 30 μL total volume four times per week (1101100). Anti-PD-1 (BioXCell BE0146) or its isotype Rat IgG2a,k (BioXCell BE0089) were given intraperitoneally at a dose of 200 μg/mouse once a week (0010000) during four weeks.

At endpoint, mice were euthanized and lungs were excised and dissociated using the Mouse Tumor Dissociation Kit (Miltenyi) and stained with conjugated antibodies to analyze the immune cell content by flow cytometry. Prior to staining, death cells were stained with the Fixable Viability Stain 510 (BD Biosciences 564406) following manufacturer's instructions. Unspecific interactions were blocked by incubation with the anti-CD16/32 antibody for 10 minutes at room temperature. For surface staining, cells were incubated with the antibodies for 20 minutes at 4° C. in the dark. The antibodies used are listed in Table 1. Cells were acquired using a CytoFlex cytometer (Beckman Coulter) and data was analyzed using the CytoExpert 2.0 software (Beckman Coulter).

The combination of Omomyc and anti-PD-1 therapy significantly increases the recruitment of CD4+ T cells expressing PD-1 but not Tim-3 to the tumor site compared to both the vehicle and anti-PD-1 only treated groups (FIG. 3). This finding indicates that the combination of Omomyc with the anti-PD-1 synergizes to promote an anti-tumor immune response. Recent findings demonstrate that PD-1 expression on tumor infiltrating lymphocytes (TILs) accurately identifies the repertoire of clonally expanded tumor-reactive cells (Gros A et al., J Clin Invest (2014) 124(5): 2246-2259). In this line of thoughts, although leading to an inhibitory signal upon ligation with its ligands (PD-L1 and PD-L2), it is now clear that PD-1 expression is first a marker of T cell activation and of high avidity TILs specific for tumor antigens (reviewed in Simon S and Labarriere N. OncoImmunology (2018). 7:1, e1364828).

Omomyc in Combination with Anti-PD-1 Induces the Production of IFN-γ

The experimental model, Omomyc and anti-PD-1 treatments are the same as the ones described above.

At endpoint, mice were euthanized and lungs were excised and dissociated using the Mouse Tumor Dissociation Kit (Miltenyi) and stained with conjugated antibodies to analyze the immune cell content by flow cytometry. Prior to staining, death cells were stained with the Fixable Viability Stain 510 (BD Biosciences 564406) following manufacturer's instructions. Unspecific interactions were blocked by incubation with the anti-CD16/32 antibody for 10 minutes at room temperature. For surface staining, cells were incubated with the antibodies for 20 minutes at 4° C. in the dark. The antibodies used are listed in Table 1. For IFN-γ staining, harvested and dissociated tumor cells were stimulated with PMA plus ionomicin (both from Sigma-Aldrich) in the presence of monensin and befeldrin A (both from BD Biosciences) for 12 hours. Cells were then harvested and stained for flow cytometry analysis. For the intracellular staining of IFN-γ, the BD Cytofix/Cytoperm buffer set was used (BD Biosciences 554722) following manufacturer's instructions. Cells were acquired using a CytoFlex cytometer (Beckman Coulter) and data was analyzed using the CytoExpert 2.0 software (Beckman Coulter). These experiments show that the combined therapy of Omomyc plus anti-PD-1 significantly induces the production of interferon-γ (IFN-γ) by both CD4+ helper and CD8+ cytotoxic intratumoral T cells (FIG. 4) compared to their vehicle counterparts, fact that was not observed either in the Omomyc nor the anti-PD-1 treated groups.

In the last few years a substantial amount of evidence has accumulated that demonstrates a critical role for IFN-γ in promoting tumor rejection and clearance. This cytokine is mainly produced by activated T and NK cells and exerts its anti-tumor effect by directly inducing anti-proliferative, pro-apoptotic and necroptotic effects on tumor cells and enhancing immunogenicity by inducing the upregulation of major histocompatibility molecules (extensively reviewed in Castro F et al., Front. Immunol. (2018). 9:847 and in Ikeda H et al., Cytokine & Growth Factors Reviews (2002). 13 95-109). Moreover, this cytokine also has an impact on the tumor microenvironment impairing angiogenesis through the inhibition of proliferation and survival of endothelial cells surrounding the tumor and thus inducing ischemia at the tumor site, an important mechanism that leads to tumor rejection (Beatty G and Paterson Y. J Immunol (2001) 166:2276-82, Kammertoens T et al., Nature (2017) 545:98-102 and Briesemeister D et al., Int J Cancer (2011) 128:371-8). Furthermore, the production of IFN-γ by Th1 CD4+ and CD8+ T cells enhances tumor clearance, as this cytokine is critical for T and NK cells trafficking to the tumor site (Melero I et al., Cancer Discov (2014) 4:522-6). In addition, IFN-γ also plays a pivotal role in activating macrophages and promoting their tumoricidal activity (Celada A et al., J Exp Med (1984) 160:55-74). Importantly, increased levels of IFN-γ is a predictive biomarker of response to chemotherapy and radiotherapy, as well as to both anti-PD-1 and CLTA-4 immunotherapies (Karachaliou N et al., Ther Adv Med Oncol (2018) 10:1758834017749748 and Mo X et al., Cancer Res (2018) 78:436-50). Consistently, recent clinical trials have already promising results showing the association between IFN-γ producing effector T cells and tumor growth inhibition (Liakou C I et al., Proc Natl Acad Sci USA (2008) 105:14987-92, Peng W et al., Cancer Res (2012) 72:5209-18 and Overacre-Delgoffe A E et al., Cell (2017) 169:1130-41.e11).

TABLE 1
Anti-mouse antibodies used for flow cytometry analysis

| | | | | Catalog |
|---|---|---|---|---|
| Antibody | Fluorochrome | Clone | Manufacturer | Number |
| CD16/32 | — | 93 | Biolegend | 101302 |
| CD45 | BV605 | 30-F11 | Biolegend | 103140 |
| CD3e | FITC | 145-2C11 | eBioscience | 11-0031 |
| CD4 | PerCP-eFluor710 | RM4-5 | eBioscience | 46-0042 |
| CD8a | APC-H7 | 53-6.7 | BD Biosciences | 560182 |
| PD-1 | BV421 | 29F.1A12 | Biolegend | 135218 |
| Tim-3 | PE-CY7 | RMT3-23 | eBioscience | 25-5870 |
| IFN-γ | APC | XMG1.2 | BD Bioscience | 554413 |

Combination of Omomyc with Anti-PD-1 Antibody Synergistically Increases the Proportion of Healthy Lung and Recruits T Cells to the Tumor Site.

KRas^{LSL-G12D/+} mice were genotyped by Transnetyx and generation of lung tumors in both males and females was performed as described previously (Jackson, E. L., et al., Analysis of lung tumor initiation and progression using conditional expression of oncogenic K-ras. Genes Dev, 2001. 15(24): p. 3243-8). Animals were maintained in a pure C57BL/6 background. After 14-16 weeks after Adeno-Cre infection, once the mice presented detectable tumors by micro-CT, they were randomized into 4 groups treated as follows for 4 weeks: Vehicle+Isotype Rat IgG2a,k, Omomyc+Isotype Rat IgG2a,k, vehicle+anti-PD-1 and Omomyc+anti-PD-1. For Omomyc treatment, animals were anesthetized by inhaled isoflurane (AbbVie Farmaceutica S.L.U.) and were intranasally treated with either the Omomyc polypeptide (3.75 mg/kg) or the vehicle (PBS pH=7) in 30 μL total volume four times per week (1101100). Anti-PD-1 (BioXCell BE0146) or its isotype Rat IgG2a,k (BioXCell BE0089) were given at 5 mg/kg intraperitoneally once a week (0010000) during four weeks.

microCT studies were acquired with a Quantum FX imaging system (Perkin Elmer. 940 Winter St. Waltham, Mass. EEUU) and image reconstruction was based on Feldkamp's method. For thorax volume, Quantum FX analysis software was used. Firstly, distance between contralateral ribs was measured at carina level (r2). Second measure was defined as the maximum distance from carina level to diaphragmatic cupule (h). Last measure was thorax height, defined as the distance between sternum and sub-lumbar musculature at diaphragmatic cupule level (r1). With these three values, truncated cone volume was calculated, using following mathematical formula:

Volume=Height*pi/3*(r13−r23)/(r1−r2)

Lung healthy tissue was calculated using a threshold method, with AMIDE software (Amide© Andreas Loening). This threshold selects the complete amount of voxels in the image that have an intensity value included in the range −950/−350 greys. This grey scale range was selected manually after examining different studies.

Finally, healthy lung/thorax volume ratios were calculated, based on the notion that complete thorax volume is maintained, while healthy lung volume is reduced gradually with pathology progression. Animals treated with Omomyc in combination with anti-PD-1 presented increased proportions of healthy lung compared to the vehicles and the treatments alone (FIGS. 5A and 5B).

At endpoint, mice were euthanized and lungs were excised and dissociated using the Mouse Tumor Dissociation Kit (Miltenyi) and stained with conjugated antibodies to analyze the immune cell content by flow cytometry. Prior to staining, death cells were stained with the Fixable Viability Stain 510 (BD Biosciences 564406) following manufacturer's instructions. Unspecific cross-reactions were blocked by incubation with the anti-CD16/32 antibody for 10 minutes at room temperature. For surface staining, cells were incubated with the antibodies for 20 minutes at 4° C. in the dark. The antibodies used are listed in Table 2.

TABLE 2
Anti-mouse antibodies used for flow cytometry analysis

| | | | | Catalog |
|---|---|---|---|---|
| Antibody | Fluorochrome | Clone | Manufacturer | Number |
| CD16/32 | — | 93 | Biolegend | 101302 |
| CD45 | BV605 | 30-F11 | Biolegend | 103140 |
| CD3e | FITC | 145-2C11 | eBioscience | 11-0031 |
| CD4 | PerCP-eFluor710 | RM4-5 | eBioscience | 46-0042 |
| CD8a | APC-H7 | 53-6.7 | BD Biosciences | 560182 |
| PD-1 | BV421 | 29F.1A12 | Biolegend | 135218 |
| Tim-3 | PE-CY7 | RMT3-23 | eBioscience | 25-5870 |
| IFN-γ | APC | XMG1.2 | BD Bioscience | 554413 |
| IL-17 | PE | TC11- | BD Bioscience | 559502 |
| | | 18H10 | | |

For IFN-γ and IL-17 staining, harvested and dissociated tumor cells were stimulated with PMA plus ionomicin (both from Sigma-Aldrich) in the presence of monensin and befeldrin A (both from BD Biosciences) for 12 hours. Cells were then harvested and stained for flow cytometry analysis. For the intracellular staining of IFN-γ, the BD Cytofix/Cytoperm buffer set (BD Biosciences 554722) was used following manufacturer's instructions. Cells were acquired using a CytoFlex cytometer (Beckman Coulter) and data were analyzed using the CytoExpert 2.0 software (Beckman Coulter).

FIG. 5C show that Omomyc and anti-PD-1 administered in combination induced T cell recruitment to the tumor site, in particular of CD4 T cells and of Th1/Th17 cells. Table 3 shows that the effect obtained is synergic. It is considered synergy when the increase in the immune cell population of interest is higher than the sum of the increase of individual treatments.

TABLE 3
Mean values of each immune cell population

| | Vehicle | Omomyc | PD-1 | Omo + PD-1 |
|---|---|---|---|---|
| % CD3 (Mean) | 15.78 | 16.61 | 16.69 | 18.62 |
| Increase vs Vehicle | | 0.83 | 0.91 | 2.84 |
| Sum of individual | | 1.74 | | Synergy |
| treatments | | | | |
| % CD4 (Mean) | 6.981 | 9.623 | 8.003 | 10.75 |
| Increase vs Vehicle | | 2.642 | 1.022 | 3.769 |
| Sum of individual | | 3.664 | | Synergy |
| treatments | | | | |
| % Th1/Th17 (Mean) | 3.424 | 6.707 | 3.148 | 8.054 |
| Increase vs Vehicle | | 3.283 | −0.276 | 4.63 |
| Sum of individual | | 3.007 | | Synergy |
| treatments | | | | |

Combination of Omomyc with Anti-CTLA-4 Antibody Synergistically Decreases Tumor Growth and Recruits Anti-Tumor T Cells to the Tumor Site

The experimental model, micro-CT scans and FACS stainings were the same as the ones described in FIG. 5.

After 14-16 weeks after Adeno-Cre infection, once the mice presented detectable tumors by micro-CT, they were randomized into 4 groups treated as follows for 4 weeks: Vehicle+Isotype Syrian hamster IgG, Omomyc+Isotype Syrian hamster IgG, vehicle+anti-CTLA-4 and Omomyc+anti-CTLA-4. For Omomyc treatment, animals were anesthetized by inhaled isoflurane (AbbVie Farmaceutica S.L.U.) and were intranasally treated with either the Omomyc polypeptide (3.75 mg/kg) or the vehicle (PBS pH=7) in 30 μL total volume four times per week (1101100). Anti-CTLA-4 (BioXCell 13E0131) or its isotype Syrian hamster IgG (BioXCell BE0087) were given at 10 mg/kg intraperitoneally once a week (0010000) during four weeks.

FIG. 6A shows that animals treated with Omomyc in combination with anti-CTLA-4 presented decreased tumor growth compared to the vehicles and the treatments alone. FIG. 6B shows that Omomyc and anti-CTLA-4 administered in combination induced T cell recruitment to the tumor site, in particular of CD4 T cells and of both CD4 and CD8 PD-1+ T cells. Table 4 shows that the effect obtained is synergic. It is considered synergy when the increase in the immune cell population of interest is higher than the sum of the increase of individual treatments.

TABLE 4
Mean values of each immune cell population

| | Vehicle | Omomyc | PD-1 | Omo + PD-1 |
|---|---|---|---|---|
| % CD3 (Mean) | 25.53 | 27.61 | 29.38 | 31.58 |
| Increase vs Vehicle | | 2.08 | 3.85 | 6.05 |
| Sum of individual | | 5.93 | | Synergy |
| treatments | | | | |
| % CD4 (Mean) | 9.394 | 10.96 | 11.97 | 15.29 |
| Increase vs Vehicle | | 1.566 | 2.576 | 5.896 |
| Sum of individual | | 4.142 | | Synergy |
| treatments | | | | |
| % CD4 + PD-1 + | 43.49 | 52.69 | 46.45 | 67.66 |
| (Mean) | | | | |
| Increase vs Vehicle | | 9.2 | 2.96 | 24.17 |
| Sum of individual | | 12.16 | | Synergy |
| treatments | | | | |
| % CD8 + PD-1 + | 35.8 | 38.87 | 42.33 | 52.5 |
| (Mean) | | | | |
| Increase vs Vehicle | | 3.07 | 6.53 | 16.7 |
| Sum of individual | | 9.6 | | Synergy |
| treatments | | | | |

Sequential Combination of Omomyc Administered Intravenously with Anti-PD-1 Antibody Synergistically Recruits Anti-Tumor T Cells to the Tumor Site

The experimental model, micro-CT scans and FACS stainings were the same as the ones described for FIG. 5.

After 14-16 weeks after Adeno-Cre infection once the mice presented detectable tumors by micro-CT, they were randomized into 4 groups treated as follows for 4 weeks: Vehicle, Omomyc, vehicle+anti-PD-1 and Omomyc+anti-PD-1. For Omomyc treatment, animals were intravenously treated with either the Omomyc polypeptide (50 mg/kg) twice per week for 10 days or the vehicle (NaAc 24 mM+150 mM NaCl) (0100100). The group receiving the combination was treated during the first 10 days twice per week with Omomyc. After the 10 days of Omomyc treatment, the group receiving the combination discontinued the Omomyc treatment and started receiving the anti-PD-1 (BioXCell BE0146) at 2.5 mg/kg intraperitoneally once a week (0010000) until the end of the experiment. The monotherapy group, Omomyc alone, received Omomyc twice per week during the first 10 days and then continued the treatment but receiving only once per week until the end of the experiment.

FIG. 7 shows that sequential treatment with Omomyc and then with anti-PD-1 induced T cell recruitment to the tumor site, in particular of CD4 T cells expressing both the PD-1 and Tim-3 molecules and of Th1/Th17 T cells expressing PD-1. Table 5 shows that the effect obtained is synergic. It is considered synergy when the increase in the immune cell population of interest is higher than the sum of the increase of individual treatments.

TABLE 5
Mean values of each immune cell population

| | Vehicle | Omomyc | PD-1 | Omo + PD-1 |
|---|---|---|---|---|
| % CD4 + PD-1 + | 3.281 | 3.5 | 2.739 | 5.442 |
| Tim-3 + (Mean) | | | | |
| Increase vs Vehicle | | 0.219 | −0.542 | 2.161 |
| Sum of individual | | −0.323 | | Synergy |
| treatments | | | | |
| % Th1/Th17 (Mean) | 0.664 | 0.615 | 0.501 | 0.994 |
| Increase vs Vehicle | | −0.049 | −0.163 | 0.33 |
| Sum of individual | | −0.212 | | Synergy |
| treatments | | | | |

Combination of Omomyc Administered Intravenously Concomitantly with Anti-PD-1 Antibody Synergistically Recruits T Cells to the Tumor Site

The very aggressive Kras/p53 mutated NSCLC MuH-163 cell line was inoculated subcutaneously (1×10⁶ cells) into C57/BL6 syngeneic mice. Once the tumors were established, mice were randomized into 4 groups: Vehicle, Omomyc, vehicle+anti-PD-1 and Omomyc+anti-PD-1. Omomyc treatment was given intravenously at 50 mg/kg (0010000) and the anti-PD-1 antibody intraperitoneally at 5 mg/kg, concomitantly once a week for 3 weeks. Mice were monitored twice a week and tumor growth was followed by caliper measurement.

At endpoint tumors were collected and half of them were fixed with 4% of PFA and embedded in paraffin wax for IHC analysis, while the other half was digested using the Mouse Tumor Dissociation Kit (Miltenyi) and stained with conjugated antibodies to analyze the immune cell content by flow cytometry. FACS staining and analysis were performed as described for FIG. 5.

For CD3 immunofluorescence, antigen retrieval was performed by heating 20 min in 0.01 M citrate buffer pH 6.0 using a microwave set at 400 W. After blocking 45 minutes in 3% BSA and washing in PBS, slides were incubated overnight at 4° C. with anti-CD3 (Dako A0452) diluted 1/100 in Dako Ready-to-use diluent (Dako S2022). After a PBS wash, slides were incubated with goat anti-rabbit IgG (H+L)-AlexaFluor®488 conjugate (Thermo Fisher Scientific A-11008) diluted 1/200 and stained with DAPI (Life Technologies D1306) diluted 1/10000, washed once with water and mounted with Fluorescence Mounting Medium (Dako S3023). CD3 positivity was measured from 5 representative fluorescent microscopy images per animal captured at 20× magnification.

FIG. 8 shows that concomitant treatment of Omomyc and anti-PD-1 significantly recruits T cells to the tumor site. Table 6 shows that the effect obtained is synergic. It is considered synergy when the increase in the immune cell population of interest is higher than the sum of the increase of individual treatments.

TABLE 6
Mean values of each immune cell population

| | Vehicle | Omomyc | PD-1 | Omo + PD-1 |
|---|---|---|---|---|
| % CD3 (Mean) | 190.3 | 303.6 | 254.3 | 368.8 |
| Increase vs Vehicle | | 113.3 | 64 | 178.5 |
| Sum of individual | | 177.3 | | Synergy |
| treatments | | | | |
| % CD45 (Mean) | 19.25 | 21.26 | 23.9 | 28.03 |
| Increase vs Vehicle | | 2.01 | 4.65 | 8.78 |
| Sum of individual | | 6.66 | | Synergy |
| treatments | | | | |

High Expression of CD3, CD4, IL-17 and IFN-γ Correlates with Higher Survival Rates

Kaplan-Meier Plots were done using the online software Kaplan-Meier Plotter (http://kmplot.com/analysis/index.php?p=background). To do so, the database of lung cancer patients was selected. All histological types of NSCLC, all stages and all grades were included for the analysis.

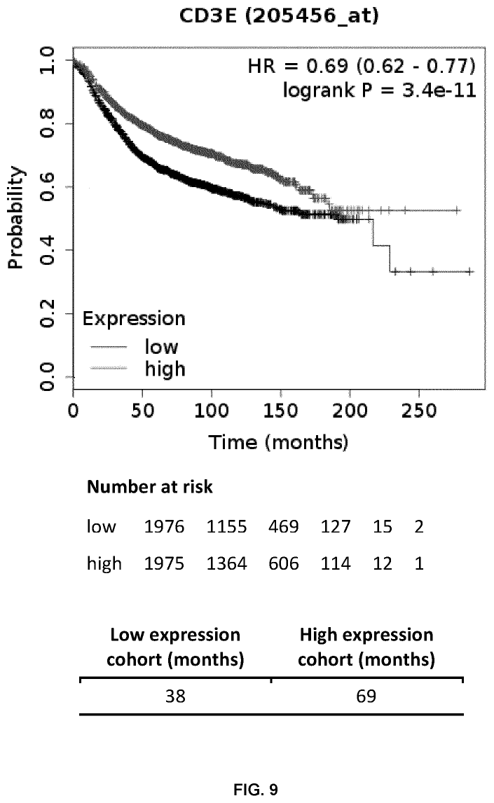

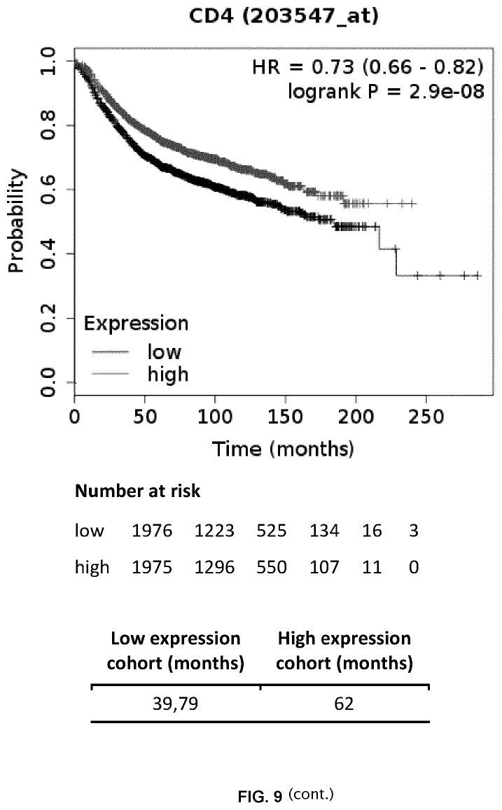

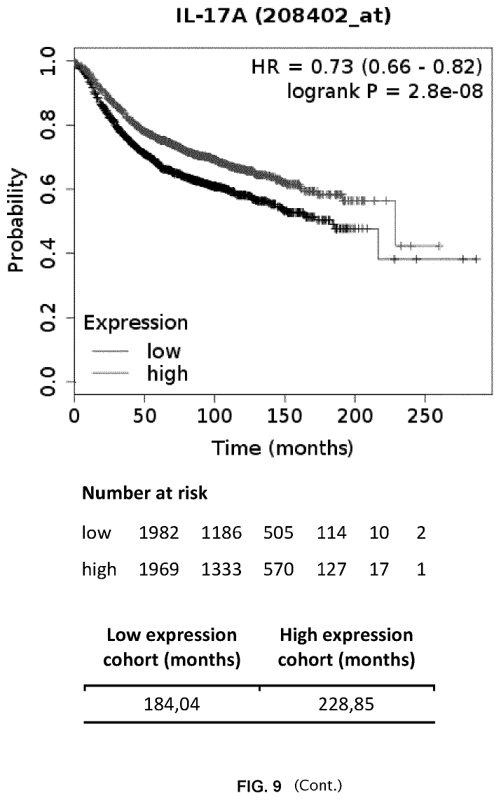

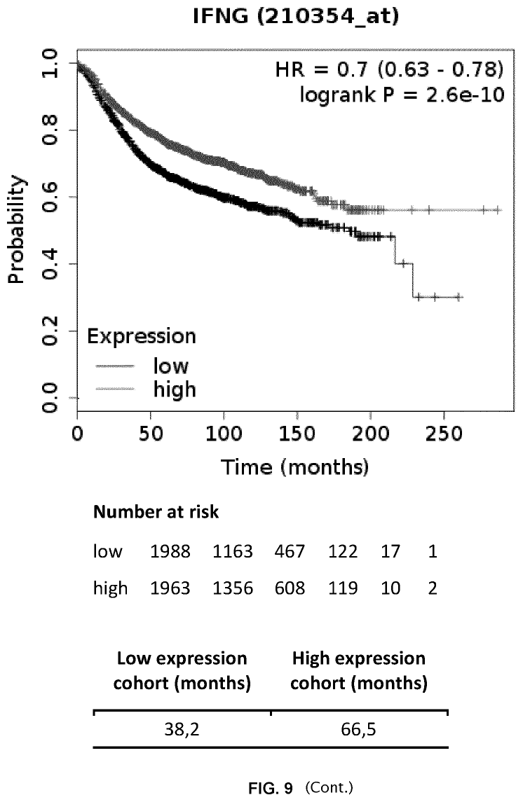

FIG. 9 shows that high expression of CD3, CD4, IL-17 and IFN-γ correlates with higher survival rates in NSCLC patients.

Discussion

The combination of intranasal Omomyc with anti-PD-1 antibody synergistically increases the proportions of healthy lung (mean 7.969) over total thorax volume, in comparison to the improvement showed by Omomyc (0.86) and anti-PD-1 (0.92) therapies alone (FIGS. 5A and 5B). In addition, the treatments administered concomitantly significantly induced the recruitment of T cells to the tumor site, in particular of CD4 T cells and the Th1/Th17 cells, which are known to exert a potent anti-tumor effect (Chatterjee, S., et al., CD38-NAD(+)Axis Regulates Immunotherapeutic Anti-Tumor T Cell Response. Cell Metab, 2018. 27(1): p. 85-100 e8) (FIG. 5C).

In line with these results, also the combination of intranasal Omomyc with anti-CTLA-4 showed decreased tumor growth (1.11) compared to the vehicle (3.85) and to both treatments administered alone (Omomyc: 2.3; a-CTLA-4: 3.0) (FIG. 6A).

In addition to this direct effect on tumor growth, the treatment combination also synergistically induced T cell recruitment to the tumor site, especially of CD4 T cells and both CD4 and CD8 T cells expressing the PD-1 molecule (cells known to identify tumor-specific T cells (Gros, A., et al., PD-1 identifies the patient-specific CD8(+) tumor-reactive repertoire infiltrating human tumors. J Clin Invest, 2014. 124(5): p. 2246-59) (FIG. 6B). In summary, intranasally administered Omomyc in combination with anti-PD-1 or anti-CTLA-4 reduces tumor growth and synergistically recruits anti-tumor T cells to the tumor site.

Furthermore, using the same mouse model (Kras^{G12D}-driven NSCLC), the inventors have demonstrated that the combination of intravenous Omomyc with anti-PD-1 administered sequentially (first Omomyc and then the anti-PD-1 antibody) also synergizes and induces the recruitment of T cells to the tumor site, especially tumor-specific CD4 expressing both PD-1 and Tim-3 molecules and Th1/Th17 anti-tumor T cells (FIG. 7).

To validate this synergistic effect in another model, the authors combined Omomyc and anti-PD-1 in another very aggressive model of NSCLC driven by mutations in both Kras and p53. Again, both drugs synergized and significantly recruited more T cells to the tumor site (FIG. 8A) and also recruited more overall immune cells (FIG. 8B).

In summary, the authors conclude that the treatment of Omomyc in combination with both anti-PD-1 and CTLA-4 therapies is able to reduce tumor growth and to synergistically recruit anti-tumor T cells to the tumor site. This therapeutic effect was observed using different routes of administration, different Omomyc doses and different doses of anti-PD-1 and CTLA-4.

This immune cell recruitment has a clear therapeutic impact in NSCLC cancer patients, as increased proportions of overall CD3 T cells, CD4 and T cells secreting IFN-g and IL-17 correlates with increased survival (FIG. 9). This evidence underlines the importance of the findings described above regarding the combination of Omomyc with an immuno-oncology agent. The same conclusion can be extrapolated to other types of cancer, where data based on immune signatures have established that a strong immune cell component is predictive of a good response to chemotherapy in breast cancer where high tumor infiltrating lymphocytes (TILs) is associated with a higher response rate to neoadjuvant therapy. In hepatic metastases of colorectal cancer, high infiltration of CD8⁺ T cells predicts a better response to chemotherapy and prolonged survival. In melanoma, the expression of an immune signature (namely, high expression of Th1 cells and cytotoxicity-associated genes) correlates with good clinical response to a therapeutic vaccine using the melanoma-associated antigen 3 (MAGEA3) (reviewed in Fridman, W. H., et al., The immune contexture in cancer prognosis and treatment. Nat Rev Clin Oncol, 2017. 14(12): p. 717-734).

In the last few years a substantial amount of evidence has accumulated that demonstrates a critical role for TILs for both tumor eradication and for efficacy of immuno-oncology therapies. In fact, a major factor involved in resistance to immuno-oncology therapies is the lack of tumor T cell infiltration, characterizing the so-called “cold tumors.” Treatment of this immune inert tumors with immuno-oncology agents represents a great challenge, since they do not display any adaptive immune response against the tumor and fail to respond to this type of therapies (Bonaventura, P., et al., Cold Tumors: A Therapeutic Challenge for Immunotherapy. Front Immunol, 2019. 10: p. 168).

Patients who never demonstrate a clinical response or stabilized disease upon PD-1/PD-L1 blockade are referred to as having “primary resistance” to therapy. In contrast, early data from clinical trials demonstrated that the presence of pre-existing TILs within the tumor and its periphery, along with co-localized PD-1 and PD-L1 expression on the T cells and tumor cells, respectively, predicted therapeutic response to anti-PD-1 therapy (Nowicki, T. S., S. Hu-Lieskovan, and A. Ribas, Mechanisms of Resistance to PD-1 and PD-L1 Blockade. Cancer J, 2018. 24(1): p. 47-53). On the same line of evidence, efficacy of Pembrolizumab (anti-PD-1) correlates with the presence of intratumoral T cells and the expression of PD-1/PD-L1 as requirements for a potent antitumor response (Tumeh, P. C., et al., PD-1 blockade induces responses by inhibiting adaptive immune resistance. Nature, 2014. 515(7528): p. 568-71) and efficacy of anti-PD-1 agents (Ribas, A., Tumor immunotherapy directed at PD-1. N Engl J Med, 2012. 366(26): p. 2517-9).

Taking all this evidence into account, it is demonstrated that the combination of Omomyc with immuno-oncology agents that synergistically induce T cell infiltration and stimulation will ultimately lead to improved clinical response rates to immuno-oncology therapies.

1-20. (canceled)

21. A combination comprising:
i) a first component selected from the group consisting of:
a) a polypeptide comprising the sequence SEQ ID NO: 1 or a functionally equivalent variant thereof,
b) a conjugate comprising a polypeptide comprising the sequence SEQ ID NO: 1 or a functionally equivalent variant thereof and a chemical moiety that facilitates cellular uptake of the polypeptide or of the functionally equivalent variant thereof,
c) a polynucleotide encoding the polypeptide of a) or the conjugate of b)
d) a vector comprising the polynucleotide according to c), and
e) a cell capable of secreting into the medium the polypeptide according to a) or the conjugate according to b).
and
ii) a second component that is an immuno-oncology agent, or
a pharmaceutical composition comprising a pharmaceutically effective amount of said combination and a pharmaceutically acceptable excipient.

22. The combination or pharmaceutical composition according to claim 21, wherein the functionally equivalent variant of SEQ ID NO: 1 is selected from the group consisting of SEQ ID NO: 4, SEQ ID NO: 5, SEQ ID NO; 6, SEQ ID NO: 7, SEQ ID NO: 8, SEQ ID NO: 9, and SEQ ID NO: 10.

23. The combination or pharmaceutical composition according to claim 21, wherein the chemical moiety that facilitates the cellular uptake of the polypeptide or the functionally equivalent variant thereof is a cell-penetrating peptide sequence and wherein said cell penetrating peptide sequence and said polypeptide or functionally equivalent variant thereof form a fusion protein.

24. The combination or pharmaceutical composition according to claim 23, wherein the cell-penetrating peptide sequence is selected from the group consisting of GRKKRRQRRR (SEQ ID NO: 37) and RRRRRRLR (SEQ ID NO: 38).

25. The combination or pharmaceutical composition according to claim 21, wherein the conjugate further comprises a nuclear localization signal.

26. The combination or pharmaceutical composition according to claim 21, wherein the immuno-oncology agent is not a cytokine.

27. The combination or pharmaceutical composition according to claim 21, wherein the immuno-oncology agent is an antagonist of a protein that inhibits T cell activation or an immune checkpoint inhibitor.

28. The combination or pharmaceutical composition according to claim 27, wherein the antagonist is selected from an anti-PD-1 agent and an anti-CTLA-4 agent.

29. The combination or pharmaceutical composition according to claim 28, wherein the antagonist is an anti-PD-1 agent.

30. The combination or pharmaceutical composition according to claim 28, wherein the antagonist is an antagonistic antibody.

31. The combination or pharmaceutical composition according to claim 30, wherein the antagonistic antibody is pembrolizumab.

32. The combination or pharmaceutical composition according to claim 21, wherein the first component is a polypeptide comprising the sequence SEQ ID NO: 1.

33. A method for the prevention and/or treatment of cancer that comprises administering to a subject in need thereof a therapeutically effective amount of the combination or pharmaceutical composition according to claim 21.

34. The method according to claim 33, wherein the cancer is lung cancer.

35. The method according to claim 33, wherein the first component is administered systemically or intranasally.

36. The method according to claim 35, wherein the intranasal administration is performed by instillation or nasal inhalation.

37. The method according to claim 33, wherein the first component is administered intranasally or intravenously and the second component is administered systemically.

38. The method according to claim 33, where T cells are recruited to the tumor site.

39. The method according to claim 33, wherein the method induces expansion of T regulatory cells.

40. The method according to claim 33, wherein the method induces production of IFN-gamma by intratumoral CD4+ and CD8+ cells.
